(19) United States
(12) Patent Application Publication
(10) Pub. No.: US 2005/0260629 A1
(43) Pub. Date: Nov. 24, 2005

(54) Differential drug sensitivity

(75) Inventor: Xin Lu, London (GB)

Correspondence Address: KLARQUIST SPARKMAN, LLP, 121 SW SALMON STREET, SUITE 1600, PORTLAND, OR, 97204, US

(73) Assignee: Ludwig Institute for Cancer Research

(21) Appl. No.: 11/077,712

(22) Filed: Mar. 10, 2005

Related U.S. Application Data
(60) Provisional application No. 60/553,879, filed on Mar. 16, 2004.

(30) Foreign Application Priority Data
Mar. 12, 2004 (GB) GB04056005.7

(51) Int. Cl. C12Q 1/68; C07H 21/04

(52) U.S. Cl. 435/6

(57) ABSTRACT

We describe a method to diagnose and treat an animal, preferably a human, suffering from a condition, typically cancer, that would benefit from a stimulation of apoptosis in tumour cells and including screening methods to identify new chemotherapeutic agents.

### CROSS REFERENCE TO RELATED APPLICATIONS

This application claims priority to U.S. Provisional Application No. 60/553,879 filed Mar. 16, 2004 and to Great Britain Application No. GB04056005.7 filed Mar. 12, 2004.

### FIELD

The invention relates to a method to diagnosis and treat an animal, preferably a human, suffering from a condition, typically cancer, that would benefit from a stimulation of apoptosis in tumour cells and including screening methods to identify new chemotherapeutic agents.

### BACKGROUND

Apoptosis is a process by which multi-cellular organisms regulate cell number and differentiation. The process is regulated by factors which either induce or prevent apoptosis. Inducers of apoptosis include Bcl-2 family members, caspase family members and their associated factors Apaf-1 and Fadd. Caspases are synthesised as proenzymes which become activated after proteolytic cleavage. The active caspase then induces many of the morphological and biochemical changes associated with apoptosis. Mitochondria play a pivotal role in the activation process through the release of pro-apoptotic factors such as cytochrome c, AIF and Diablo. The release of factors from mitochondria is controlled by the Bcl-2 family of proteins; (such as Bcl-2 and Bcl-x1 inhibit release; Bax and Bak induce release).

Tumour suppressor proteins have pro-apoptotic activities. Tumour suppressor genes encode proteins which function to inhibit cell growth or division and are therefore important with respect to maintaining proliferation, growth and differentiation of normal cells. Mutations in tumour suppressor genes result in abnormal cell-cycle progression whereby the normal cell-cycle check points which arrest the cell-cycle, when, for example, DNA is damaged, are ignored and damaged cells divide uncontrollably. The products of tumour suppressor genes function in all parts of the cell (such as cell surface, cytoplasm, and nucleus) and prevent the passage of damaged cells through the cell-cycle.

Arguably the tumour suppressor gene which has been the subject of the most intense research is p53. p53 encodes a protein which functions as a transcription factor and is a key regulator of the cell division cycle. It was discovered in 1978 as a protein shown to bind with affinity to the SV40 large T antigen. The p53 gene encodes a 393 amino acid polypeptide with a molecular weight of 53 kDa. Genes regulated by the transcriptional activity of p53 contain a p53 recognition sequence in their 5′ regions. These genes are activated when the cellular levels of p53 are elevated due to, for example, DNA damage. Examples of genes that respond to p53 include, mdm2, Bax and PIG-3. Bax and PIG-3 are involved in one of the most important functions of p53, the induction of apoptosis.

Recently, a new protein family, specifically shown to modulate the apoptotic pathway of p53, has been discovered. This family, named ASPP for Apoptosis Stimulating Protein of p53, is composed of three members. ASPP1 and ASPP2 interact with p53 and specifically enhance p53-dependent transcription of apoptotic genes, such as Bax or Pig3 as well as synergize with p53 to induce apoptosis (Samuels-Lev et al., Mol. Cell. 8:781-94, 2001). The C-terminus of ASPP2 was initially isolated from a yeast two-hybrid assay as a p53 interacting protein (53BP2) (Iwabuchi et al., Proc. Natl. Acad. Sci. USA. 91:6098-102, 1994). The crystal structure of the 53BP2-p53 complex showed that 53BP2 binds p53 in its DNA binding domain, as does the large T antigen of simian virus 40 (Gorina and Pavletich, Science 274:1001-5, 1996). 53BP2 was subsequently discovered to interact with other proteins including protein phosphatase 1 (PP1) (Helps et al., FEBS Lett. 377:295-300, 1995), Bcl-2 (Naumovski and Cleary, Mol. Cell. Biol. 16:3884-92, 1996) and NF-KB subunit p65 (Yang et al., Oncogene 18:5177-86, 1999).

The protein was renamed bBp2 and found to be 1005 amino acids from in vitro translation data (Naumovski and Cleary, Mol. Cell. Biol. 16:3884-92, 1996), with additional sequence N terminal to the already known proline-rich region, ankyrin repeats and SH3 domains. Using antibodies raised against bBP2 it was found that the endogenous protein is larger (1128 amino acids) and more active than bBP2. This full-length protein was renamed ASPP2. Database searching revealed a similar protein containing the proline-rich region, the ankyrin repeats and the SH3 domains; this protein, which is a longer version of a KIAA EST clone, was named ASPP1 (1090 amino acids). ASPP1 and ASPP2 have recently been shown to be activators of p53 family members binding to the p53 DNA binding domain and activating apoptosis (Bergamaschi et al., Mol. Cell. Biol. 24:1341-50, 2004).

The third member of the ASPP family is an inhibitor of ASPP1 and ASPP2 named iASPP (Bergamaschi et al., Nat. Genet. 33:162-7, 2003). iASPP was previously identified as a p65 rel A binding protein (RAI) of 315 amino acids. It is encoded by PPP1R13L in humans and ape-1 in C. elegans and was shown to be a specific inhibitor of p53 function. iASPP appears to be the most conserved member of the family since it shares 38% amino acid identity with Ce-iASPP. Structurally, iASPP contains an ankyrin repeat domain and an SH3 domain essential for the binding to p53, as in its counterparts ASPP1 and ASPP2. Most of the p53 contact residues of ASPP2 are identical in iASPP and some of them are conserved in Ce-iASPP. The similarity between the p53-binding regions of ASPP and iASPP implies a competition for interaction with p53, which suggests an important biological mechanism in the activation of the p53-dependent apoptotic pathway. Co-expression of iASPP stimulated Ras-mediated transformation by 15 fold. Moreover, from 40 human breast-tumor samples expressing wild type p53, iASPP was found overexpressed in eight tumors with normal levels of ASPP1 and ASPP2 for seven of them (Bergamaschi et al., Nat. Genet. 33:162-7, 2003). Taken together these data suggest that iASPP is an oncoprotein.

Chemotherapeutic agents are effective at killing cancer cells when compared to normal cells. Examples of these agents are well known in the art, some of which induce apoptosis. For example, etoposide and camptothecin are inhibitors of topoisomerases. As a consequence, DNA replication or DNA repair processes are blocked.

Doxorubicin and daunorubicin are DNA intercalators. Doxorubicin has been reported to induce CD95 (Fas/Apo-1) gene expression in a p53-dependent mechanism in human primary endothelial cells (Lorenzo et al., J. Biol. Chem. 277:10883-92, 2002). Moreover it has been shown to trigger apoptosis in various cell lines (Barry et al., Biochem. Pharmacol. 40:2353-62, 1990) and its application in cancer treatment has revealed that p53 accumulates in cells exposed to doxorubicin. Very little is known about the mechanistic action of daunorubicin in cells even though it has been shown to be an efficient drug in therapy (Ohnuma et al., Cancer Res. 35:1767-72, 1975).

Platinum containing compounds, for example cisplatin, carboplatin, oxaloplatin, are known to have anti-cancer activity. Cisplatin is a neutral inorganic component, which interacts with nucleophilic N7-sites of purine bases in DNA after aquation reactions to form DNA-protein and DNA-DNA interstrand and intrastrand crosslinks (Eastman, Biochem. Pharmacol. 36:4177-8, 1987). When cisplatin enters cells it is potentially vulnerable to cytoplasmic inactivation by intracellular components. Cisplatin acts on ATR and Chk2 to induce the apoptotic pathway through a p53-dependent pathway. However, p73 itself is also activated by cisplatin to trigger apoptosis. Among its other effects cisplatin is known to be responsible for the induction of cell cycle arrest through p21 (for review see (Siddik, Oncogene 22:7265-7, 2003)).

A further example of a chemotherapeutic agent is 5′Fluorouracil (5-FU). 5-FU is an antimetabolite drug widely used in treatment of colorectal cancer. 5-FU exerts its anticancer effects through inhibition of thymidylate synthase and incorporation of its metabolites into mRNA and DNA resulting into the blockage of their synthesis. Studies on 5FU have shown a clear role for p53 in cell culture, where the loss of p53 function reduces cellular sensitivity to 5FU (Longley et al., Cancer Res. 62:2644-9, 2002), and in vivo, where a number of clinical studies have found that p53 overexpression correlates with resistance to 5FU (Liang et al., Int. J. Cancer 97:451-7, 2002). Notably a microarray study has shown that FAS is a target gene for 5FU (Maxwell et al., Cancer Res. 63:4602-6, 2003). Typically, when 5FU is administered to a patient, leucovorin is also administered since it enhances the activity of agents such as 5FU.

### SUMMARY

We have surprisingly found that cells expressing ASPP family members are differentially sensitive to chemotherapeutic agents that are dependent on the expression level of ASPP½ and/or iASPP. Moreover, the p53 polymorphic form expressed by the cell also influences the response of cells to chemotherapeutic agents. A number of studies have been carried out to investigate the relationship between a common polymorphism of p53 at codon 72 (Arg/Pro) and cancer susceptibility. However the existing conclusions are controversial due to a lack of understanding of how and why the two p53 polymorphism variants function differently. In our currently unpublished application GB0328047.6, which is incorporated by reference, we show that the apoptotic function of the two polymorphic p53 variants depends on their ability to interact with the ASPP family of proteins. ASPP1 and ASPP2 selectively interact with and stimulate the apoptotic function of p53Pro72 while iASPP selectively bind and inhibit the apoptotic function of p53Pro72.

These observations provide the basis for a diagnostic assay to determine the susceptibility of a patient suffering from cancer to a particular treatment regime and the ability to implement a treatment regime with increased efficacy with respect to inducing apoptosis in tumour cells. The response of ASPP½ and iASPP also provides a means to identify new agents with chemotherapeutic activity.

According to an aspect of the invention there is provided a method to diagnose and treat an animal, preferably a human, suffering from a condition which would benefit from a stimulation of apoptosis with a chemotherapeutic agent comprising the steps of:
i) providing an isolated cell/tissue sample to be tested;
ii) determining the expression pattern of at least one nucleic acid molecule selected from the group consisting of:
a) a nucleic acid molecule comprising a nucleic acid sequence as r represented in FIG. 6A or 6B;
b) a nucleic acid molecule which hybridises under stringent hybridisation conditions to the nucleic acid molecule in (a) and which encodes a polypeptide which stimulates the apoptotic activity of p53;
c) a nucleic acid molecule consisting of a nucleic acid sequence which is degenerate as a result of the genetic code to a nucleic acid molecule as defined in (a) and (b); and
iii) administering at least one chemotherapeutic agent which stimulates the expression of a nucleic acid molecule as defined in (ii) above.
   Hybridisation of a nucleic acid molecule occurs when two complementary nucleic acid molecules undergo an amount of hydrogen bonding to each other. The stringency of hybridisation can vary according to the environmental conditions surrounding the nucleic acids, the nature of the hybridisation method, and the composition and length of the nucleic acid molecules used. Calculations regarding hybridisation conditions required for attaining particular degrees of stringency are discussed in Sambrook et al., Molecular Cloning: A Laboratory Manual (Cold Spring Harbor Laboratory Press, Cold Spring Harbor, N.Y., 2001); and Tijssen, Laboratory Techniques in Biochemistry and Molecular Biology-Hybridisation with Nucleic Acid Probes Part I, Chapter 2 (Elsevier, New York, 1993). The Tₘ is the temperature at which 50% of a given strand of a nucleic acid molecule is hybridised to its complementary strand. The following is an exemplary set of hybridisation conditions and is not limiting:
   Very High Stringency (Allows Sequences that Share at Least 90% Identity to Hybridise)
Hybridisation: 5×SSC at 65° C. for 16 hours
Wash twice: 2×SSC at room temperature (RT) for 15 minutes each
Wash twice: 0.5×SSC at 65° C. for 20 minutes each
   High Stringency (Allows Sequences that Share at Least 80% Identity to Hybridise)
Hybridisation: 5×-6×SSC at 65° C.-70° C. for 16-20 hours
Wash twice: 2×SSC at RT for 5-20 minutes each
Wash twice: 1×SSC at 55° C.-70° C. for 30 minutes each
   Low Stringency (Allows Sequences that Share at Least 50% Identity to Hybridise)
Hybridisation: 6×SSC at RT to 55° C. for 16-20 hours
Wash at least twice: 2×-3×SSC at RT to 55° C. for 20-30 minutes each.

In a preferred embodiment of the invention said cell/tissue sample comprises a cancer cell. Preferably said cell/tissue sample is a breast cell/tissue sample.

In a further preferred method of the invention said cell/tissue sample is further analysed to determine the p53 genotype of said animal.

In a preferred method of the invention said p53 genotype is a p53 variant wherein said variant is modified by substitution of an amino acid residue encoded by codon 72 of the nucleic acid sequence as represented in FIG. 7.

In a preferred method of the invention said p53 variant varies at codon 72 wherein said codon encodes a proline amino acid residue.

In a preferred method of the invention said p53 variant varies at codon 72 wherein said codon encodes an arginine amino acid residue.

In a further preferred method of the invention the said cell/tissue sample is further analysed to determine the expression of a nucleic acid molecule selected from the group consisting of:
i) a nucleic acid molecule comprising a nucleic acid sequence as represented in FIG. 8;
ii) a nucleic acid molecule which hybridises under stringent hybridisation conditions to the nucleic acid molecule in (i) and which encodes a polypeptide which inhibits the apoptotic activity of p53;
iii) a nucleic acid molecule consisting of a nucleic acid sequence which is degenerate as a result of the genetic code to a nucleic acid molecule as defined in (i) and (ii).

In a preferred embodiment of the invention said chemotherapeutic drug is a DNA intercalating drug or a topoisomerase inhibitor.

In a preferred method of the invention said chemotherapeutic drug is an anthracyline antibiotic. Preferably said agent is doxorubicin or daunorubicin, or structural variants thereof.

In a preferred method of the invention said chemotherapuetic agent is an anti-metabolic drug.

In an alternative preferred method of the invention said chemotherapeutic agent is 5-fluorouracil.

In a preferred method of the invention said anti-metabolic drug is administered with leucovorin.

In a preferred method of the invention a combined preparation of at least two different chemotherapeutic drugs are administered to said animal.

In a further preferred method of the invention said animal is administered an antagonistic agent which inhibits the activity of a polypeptide encoded by a nucleic acid molecule selected from the group consisting of:
i) a nucleic acid molecule comprising a nucleic acid sequence as represented in FIG. 8;
ii) a nucleic acid molecule which hybridises under stringent hybridisation conditions to the nucleic acid molecule in (i) and which encodes a polypeptide which inhibits the apoptotic activity of p53;
iii) a nucleic acid molecule consisting of a nucleic acid sequence which is degenerate as a result of the genetic code to a nucleic acid molecule as defined in (i) and (ii).

In a preferred embodiment of the invention said agent is an antibody, or active binding fragment thereof, that binds and inhibits the activity of said polypeptide. Preferably said antibody or active binding fragment, is a monoclonal antibody.

Antibodies or immunoglobulins (Ig) are a class of structurally related proteins consisting of two pairs of polypeptide chains, one pair of light (L) (low molecular weight) chain (κ or λ), and one pair of heavy (H) chains (γ, α, μ, δ and ε), all four linked together by disulphide bonds. Both H and L chains have regions that contribute to the binding of antigen and that are highly variable from one Ig molecule to another.

In addition, H and L chains contain regions that are non-variable or constant. The L chains consist of two domains. The carboxy-terminal domain is essentially identical among L chains of a given type and is referred to as the “constant” (C) region. The amino terminal domain varies from L chain to L chain and contributes to the binding site of the antibody. Because of its variability, it is referred to as the “variable” (V) region. The variable region contains complementarity determining regions or CDR's which form an antigen binding pocket. The binding pockets comprise H and L variable regions which contribute to antigen recognition. It is possible to create single variable regions, so called single chain antibody variable region fragments (scFv's). If a hybridoma exists for a specific monoclonal antibody it is well within the knowledge of the skilled person to isolate scFv's from mRNA extracted from said hybridoma via RT PCR. Alternatively, phage display screening can be undertaken to identify clones expressing scFv's. Alternatively said fragments are “domain antibody fragments”. Domain antibodies are the smallest binding part of an antibody (approximately 13 kDa). Examples of this technology is disclosed in U.S. Pat. No. 6,248,516, U.S. Pat. No. 6,291,158, U.S. Pat. No. 6,127,197 and EP0368684 which are all incorporated by reference in their entirety.

In a preferred method of the invention said antibody fragment is a single chain antibody variable region fragment.

In a further preferred embodiment of the invention said antibody is a humanised or chimeric antibody.

A chimeric antibody is produced by recombinant methods to contain the variable region of an antibody with an invariant or constant region of a human antibody. A humanised antibody is produced by recombinant methods to combine the complementarity determining regions (CDRs) of an antibody with both the constant (C) regions and the framework regions from the variable (V) regions of a human antibody. Chimeric antibodies are recombinant antibodies in which all of the V-regions of a mouse or rat antibody are combined with human antibody C-regions. Humanised antibodies are recombinant hybrid antibodies which fuse the complimentarily determining regions from a rodent antibody V-region with the framework regions from the human antibody V-regions. The C-regions from the human antibody are also used. The complimentarily determining regions (CDRs) are the regions within the N-terminal domain of both the heavy and light chain of the antibody to where the majority of the variation of the V-region is restricted. These regions form loops at the surface of the antibody molecule. These loops provide the binding surface between the antibody and antigen.

In a further preferred embodiment of the invention said iASPP antagonist is an RNAi molecule or an antisense molecule designed with reference to the nucleic acid sequence presented in FIG. 8.

As used herein, the term “antisense molecule” or “antisense” describes an oligonucleotide that is an oligoribonucleotide, oligodeoxyribonucleotide, modified oligoribonucleotide, or modified oligodeoxyribonucleotide which hybridises under physiological conditions to DNA comprising a particular gene or to an mRNA transcript of that gene and, thereby, inhibits the transcription of that gene and/or the translation of that mRNA. The antisense molecules are designed so as to interfere with transcription or translation of a target gene upon hybridisation with the target gene or transcript. Those skilled in the art will recognise that the exact length of the antisense oligonucleotide and its degree of complementarity with its target will depend upon the specific target selected, including the sequence of the target and the particular bases which comprise that sequence. It is preferred that the antisense oligonucleotide be constructed and arranged so as to bind selectively with the target under physiological conditions, (for example to hybridise substantially more to the target sequence than to any other sequence in the target cell under physiological conditions). Based upon the iASPP nucleic acid sequences provided herein, or upon allelic or homologous genomic and/or cDNA sequences, one of skill in the art can easily choose and synthesise any of a number of appropriate antisense molecules for use in accordance with the present invention. For example, a “gene walk” comprising a series of oligonucleotides of 15-30 nucleotides spanning the length of iASPP nucleic acid can be prepared, followed by testing for inhibition of the corresponding I expression. Optionally, gaps of 5-10 nucleotides can be left between the oligonucleotides to reduce the number of oligonucleotides synthesised and tested.

In order to be sufficiently selective and potent for inhibition, such antisense oligonucleotides should comprise at least 10 and, more preferably, at least 15 consecutive bases which are complementary to the target, although in certain cases modified oligonucleotides as short as 7 bases in length have been used successfully as antisense oligonucleotides (Wagner et al., Nature Biotechnol. 14:840-844, 1996). Most preferably, the antisense oligonucleotides comprise a complementary sequence of 20-30 bases. Although oligonucleotides may be chosen which are antisense to any region of the gene or mRNA transcripts, in preferred embodiments the antisense oligonucleotides correspond to N-terminal or 5′ upstream sites such as translation initiation, transcription initiation or promoter sites. In addition, 3′-untranslated regions may be targeted. Targeting to mRNA splicing sites has also been used in the art but may be less preferred if alternative mRNA splicing occurs. In addition, the antisense is targeted, preferably, to sites in which mRNA secondary structure is not expected (see, for example, Sainio et al., Cell Mol. Neurobiol. 14(5):439-457, 1994) and at which proteins are not expected to bind. Finally, although iASPP cDNA sequences are disclosed herein, one of ordinary skill in the art may easily derive the genomic DNA corresponding to these cDNAs. Thus, the present invention also provides for antisense oligonucleotides which are complementary to iASPP genomic DNA. Similarly, antisense to allelic or homologous cDNAs and genomic DNAs are enabled without undue experimentation.

In one set of embodiments, the antisense oligonucleotides of the invention may be composed of “natural” deoxyribonucleotides, ribonucleotides, or any combination thereof. That is, the 5′ end of one native nucleotide and the 3′ end of another native nucleotide may be covalently linked, as in natural systems, via a phosphodiester internucleoside linkage. These oligonucleotides may be prepared by art recognised methods which may be carried out manually or by an automated synthesiser. They also may be produced recombinantly by vectors.

A recent technique to specifically ablate gene function is through the introduction of double stranded RNA, also referred to as inhibitory RNA (RNAi), into a cell that results in the destruction of mRNA complementary to the sequence included in the RNAi molecule. The RNAi molecule comprises two complementary strands of RNA (a sense strand and an antisense strand) annealed to each other to form a double stranded RNA molecule. The RNAi molecule is typically derived from exonic or coding sequence of the gene which is to be ablated.

Recent studies suggest that RNAi molecules ranging from 100-1000 bp derived from coding sequence are effective inhibitors of gene expression. Surprisingly, only a few molecules of RNAi are required to block gene expression which implies the mechanism is catalytic. The site of action appears to be nuclear as little if any RNAi is detectable in the cytoplasm of cells indicating that RNAi exerts its effect during mRNA synthesis or processing.

In a preferred embodiment of the invention there is provided a transcription cassette comprising a nucleic acid sequence operatively linked to a promoter which promoter transcribes said nucleic acid molecule to produce an antisense nucleic acid molecule, said sequence selected from the group consisting of:
i) a nucleic acid sequence, or part thereof, as represented in FIG. 8;
ii) a nucleic acid sequence which hybridises under stringent hybridisation conditions to the sense sequence presented in FIG. 8 and which encodes a polypeptide with anti-apoptotic activity.

In a preferred embodiment of the invention said cassette is part of a vector.

In a further preferred embodiment of the invention there is provided a transcription cassette comprising a nucleic acid molecule or part thereof, selected from the group consisting of:
i) a nucleic acid molecule represented by the nucleic acid sequence in FIG. 8;
ii) a nucleic acid molecule which hybridises under stringent hybridisation conditions to the sequence in (i) above and which encodes a polypeptide with anti-apoptotic activity; or
iii) a nucleic acid molecule which is degenerate because of the genetic code to the sequences defined in (i) and (ii) above; wherein said cassette is adapted such that both sense and antisense nucleic acid molecules are transcribed from said cassette.

In a preferred embodiment of the invention said cassette is provided with at least two promoters adapted to transcribe both sense and antisense strands of said nucleic acid molecule.

In a further preferred embodiment of the invention said cassette comprises a nucleic acid molecule wherein said molecule comprises a first part linked to a second part wherein said first and second parts are complementary over at least part of their sequence and further wherein transcription of said nucleic acid molecule produces an RNA molecule which forms a double stranded region by complementary base pairing of said first and second parts.

In a preferred embodiment of the invention said first and second parts are linked by at least one nucleotide base.

In a preferred embodiment of the invention said first and second parts are linked by 2, 3, 4, 5, 6, 7, 8, 9 or at least 10 nucleotide bases.

In a further preferred embodiment of the invention the length of the RNAi molecule is between 100 bp-1000 bp. More preferably still the length of RNAi is selected from 100 bp; 200 bp; 300 bp; 400 bp, 500 bp; 600 bp; 700 bp; 800 bp; 900 bp; or 1000 bp. More preferably still said RNAi is at least 1000 bp.

In an alternative preferred embodiment of the invention the RNAi molecule is between 15 bp and 25 bp, preferably said molecule is 21 bp.

In a preferred embodiment of the invention said cassette is part of a vector.

In a further preferred method of the invention said treatment includes the additional administration of p53.

In a preferred method of the invention p53 is administered as a nucleic acid molecule as represented by the nucleic acid sequence shown in FIG. 7, or a nucleic acid molecule that hybridises under stringent hybridisation conditions to the nucleic acid sequence in FIG. 7 and encodes a polypeptide with the specific activity associated with p53.

In a preferred embodiment of the invention said nucleic acid molecule encodes a p53 polymorphic variant wherein said variant is modified at codon 72. Preferably said variant is p53Arg72. Alternatively said p53 variant is p53Pro72.

Preferably said nucleic acid molecule is part of a vector adapted for the expression of p53.

According to a further aspect of the invention there is provided a screening method to identify a chemotherapeutic agent that stimulates the expression of a nucleic acid molecule selected from the group consisting of:
a) a nucleic acid molecule comprising a nucleic acid sequence as represented in FIG. 6A or 6B;
b) a nucleic acid molecule which hybridises under stringent hybridisation conditions to the nucleic acid molecule in (a) and which encodes a polypeptide which stimulates the apoptotic activity of p53;
c) a nucleic acid molecule consisting of a nucleic acid sequence which is degenerate as a result of the genetic code to a nucleic acid molecule as defined in (a) and (b); comprising
i) providing a preparation comprising a vector which vector is adapted for the expression of a detectable reporter molecule wherein the expression of said reporter molecule is controlled by a promoter sequence which promoter sequence naturally controls the expression of a nucleic acid molecule as defined in (a), (b) or (c) above and at least one candidate agent to be tested; and
ii) determining the expression of said reporter molecule in the presence of said candidate agent.

“Promoter” is an art recognised term and, for the sake of clarity, includes the following features which are provided by example only. Enhancer elements are cis acting nucleic acid sequences often found 5′ to the transcription initiation site of a gene (enhancers can also be found 3′ to a gene sequence or even located in intronic sequences). Enhancers function to increase the rate of transcription of the gene to which the enhancer is linked. Enhancer activity is responsive to trans acting transcription factors (polypeptides) which have been shown to bind specifically to enhancer elements. The binding/activity of transcription factors (please see Eukaryotic Transcription Factors, by David S Latchman, Academic Press Ltd, San Diego) is responsive to a number of physiological/environmental cues which include, by example and not by way of limitation, intermediary metabolites or environmental effectors. Promoter elements also include so called TATA box and RNA polymerase initiation selection sequences which function to select a site of transcription initiation. These sequences also bind polypeptides which function, inter alia, to facilitate transcription initiation selection by RNA polymerase. Vector adaptations also include the provision of selectable markers and autonomous replication sequences which facilitate the maintenance of said vector in either the eukaryotic cell or prokaryotic host. Vectors which are maintained autonomously are referred to as episomal vectors. Vectors may also include “reporter” genes which facilitate the detection of expression from the vector. For example green fluorescent protein (GFP). Fluorescent proteins can be used to measure promoter activity in a cell without the need for lysing the cell. Fluorescence emission spectrum shifted derivatives of GFP may include blue fluorescent protein (BFP) and yellow fluorescent protein (YFP). Other derivatives include enhanced cyan yellow protein (ECYP), EYFP, EGFP. An advantage of using GFP or derivatives thereof is that two or more reporter proteins expressed in the same cell can be assayed using the same assay technique for example assaying for a particular fluorescence emission. Alternatively, the detectable marker is an; enzyme, for example, glucuronidase, luciferase. Other reporter proteins may include lac Z, and CAT.

These adaptations are well known in the art. There is a significant amount of published literature with respect to expression vector construction and recombinant DNA techniques in general. Please see, Sambrook et al (1989) Molecular Cloning: A Laboratory Manual, Cold Spring Harbour Laboratory, Cold Spring Harbour, NY and references therein; Marston, F (1987) DNA Cloning Techniques: A Practical Approach Vol III IRL Press, Oxford UK; DNA Cloning: F M Ausubel et al, Current Protocols in Molecular Biology, John Wiley & Sons, Inc. (1994).

In a preferred method of the invention said preparation comprises a cell transfected with said vector; preferably a eukaryotic mammalian cell.

In a preferred method of the invention said cell is human

In a preferred method of the invention said cell is a cancer cell, preferably a breast cancer cell.

In a further preferred method of the invention said promoter sequence comprises a nucleic acid molecule as shown in FIG. 9A or 9B, or a variant sequence wherein said sequence has been modified by addition, deletion or substitution of at least one nucleotide base and which variant substantially retains the transcriptional activity of the promoter sequence shown in FIG. 9A or 9B or has enhanced activity when compared to the unmodified promoter sequence.

In a preferred method of the invention said candidate agent is a DNA intercalating drug.

In a preferred method of the invention said candidate agent is an anti-metabolic drug.

According to a further aspect of the invention there is provided a vector comprising a promoter sequence which sequence comprises a nucleic acid molecule as shown in FIG. 9A or 9B, or a variant sequence wherein said sequence has been modified by addition, deletion or substitution of at least one nucleotide base, and which variant substantially retains the transcriptional activity of the promoter sequence shown in FIG. 9A or 9B, or has enhanced activity when compared to the unmodified promoter sequence.

According to a further aspect of the invention there is provided a non-human transgenic animal characterised in that said animal comprises at least one vector according to the invention.

It will be apparent to the skilled artisan that although the initial screening of chemotherapeutic agents would typically be a cell based assay, it would also be desirable to determine the activity of candidate agents in vivo. This could be achieved by using a non-human transgenic animal transfected with a vector as herein described. Methods to generate transgenic animals are well known in the art.

An embodiment of the invention will now be described by example only and with reference to the following Figures.

### BRIEF DESCRIPTION OF THE FIGURES

FIGS. 1A and 1B illustrate the response of the proapoptotic promoter Pig3 to various chemotherapeutic agents;

FIGS. 2A and 2B illustrate the response of the proapoptotic promoter Pig3 to doxorubicin and cisplatin; FIG. 2C illustrates that doxorubicin and daunorubicin act in a p53 dependent manner; FIG. 2D illustrates that doxorubicin and daunorubicin specifically induce a p53 dependent apoptotic pathway;

FIG. 3A illustrates that doxorubicin and cisplatin have a p53 dependent effect; FIG. 3B illustrates that doxorubicin induces p53 activity via ASPP1 and ASPP2; FIG. 3C illustrates that p53 is inhibited by iASPP in the presence of cisplatin;

FIGS. 4A and 4B illustrate that doxorubicin activates the ASPP2 promoter; FIGS. 4C and 4D illustrates that 5-fluorouracil activates the ASPP1 promoter;

FIGS. 5A and 5B illustrates the effect of titration of various chemotherapeutic drugs on the transcriptional activity of the ASPP1 and ASPP2 promoters respectively;

FIG. 6A is the nucleic acid sequence of human ASPP1 (SEQ ID NO: 1); FIG. 6B is the nucleic acid sequence of ASPP2 (SEQ ID NO: 2);

FIG. 7A is the nucleic acid sequence of human p53 (SEQ ID NO: 3); FIG. 7B is the amino acid sequence of p53 (SEQ ID NO: 4);

FIG. 8 is the nucleic acid sequence of human iASPP (SEQ ID NO: 5); and

FIG. 9A is the nucleic acid sequence of the ASPP1 promoter (SEQ ID NO: 6); FIG. 9B is the nucleic acid sequence of the ASPP2 promoter (SEQ ID NO: 7).

### DETAILED DESCRIPTION OF MULTIPLE EMBODIMENTS

Materials and Methods

Chemicals

All chemicals, unless otherwise stated, were obtained from BDH Chemicals, UK. All autoradiography films (Hyperfilm), ECL (Enhanced Chemi-Luminescence) reagents were purchased from Amersham Pharmacia Biotech (UK). All restriction enzymes, their buffers, were purchased from New England Biolabs (UK). All tissue culture dishes and flasks were from Greiner (UK).

The Luciferase Assay System Kit was purchased from Promega (WI, USA). For all DNA preparation the QIAGEN Plasmid Mega Kit (Qiagen UK) was used.

All the chemotherapy drugs were purchased from St Mary's Hospital pharmacy.

Antibodies

| Antigen | Antibody Name | Species | Source |
|---|---|---|---|
| P53 | DO-1 | Mouse mAb | Hybridoma |
| PCNA | PC-10 | Mouse mAb | Hybridoma |
| P21 | SX-118 | Mouse mAb | Serum |
| Bax | N-20 | Rabbit pAb | Santa Cruz (CA, USA) |
| ASPP2 | DX54.10 | Mouse mAb | Hybridoma |
| ASPP1 | LX054.1 | Mouse mAb | Serum |
| IASPP | LX049.3 | Mouse mAb | Serum |
| Pig3 | N-20 | Goat pAb | Santa Cruz (CA, USA) |

Abbreviations - monoclonal antibody (mAb), polyclonal antibody (pAb).

Plasmids

| Plasmid | | Source/ |
|---|---|---|
| Name | Relevant Information | Reference: |
| PGL3 | p53-responsive mdm2 (P2) promoter | Karen Vousden |
| mdm2-luc | (intron 1) linked to luciferase reporter | (original from |
| | | Zauberman et |
| | | al., 1995) |
| P21- | p53-responsive p21 promoter linked to | Bert |
| luc | luciferase reporter | Vogelstein |
| PIG3- | P53-responsive PIG3 promoter | Matthias |
| 17mer-luc | containing 17repeats of p53 binding | Dobbelstein |
| | sites linked to luciferase reporter | |
| PCDNA3.1/ | Empty vector containing CMV and 7 | Invitrogen |
| V5-His- | promoter sites | |
| TOPO ® | | |
| PCDNA3.1 | Human ASPP2 in reverse orientation | Giuseppe |
| antisense | driven by the CMV promoter and has | Trigiante |
| ASPP2 | the T7 promoter | |
| PCDNA3.1 | Human ASPP1 in reverse orientation | Giuseppe |
| antisense | driven by the CMV promoter and has | Trigiante |
| ASPP1 | the T7 promoter | |
| Si-iASPP | 19 human iASPP nucleotides cloned | Giuseppe |
| | into the pSUPER vector | Trigiante |
| | (Brummelkamp et al., 2002) generating | |
| | a RNAi | |
| pCDNA3 V5 | Two V5 epitopes were cloned in | Invitrogen/ |
| tagged | repeat into the polylinker of the | Susana |
| | pCDNA3 vector between BamHI and | Llanos |
| | EcoRI. | |
| V5-iASPP | iASPP cDNA tagged in its N-terminal | Invitrogen/ |
| | part and cloned into pCDNA3 V5 | Susana |
| | tagged vector between the EcoRI and | Llanos |
| | XhoI restriction sites. | |
| iASPP-V5 | iASPP cDNA tagged in its C-terminal | Invitrogen/ |
| | and cloned using TOPO ® cloning | Susana |
| | technique. | Llanos |

Cell lines

| Name | Tissue Type/Origin |
|---|---|
| MCF7 | Human Breast, wild type p53 |
| U2OS | Human osteosarcoma, wild type p53 |

Constructed cell lines

| Name | Description |
|---|---|
| Luciferase stable | Human osteosarcoma, wild type p53, stably co- |
| cell lines in U2OS | transfected with either Pig3-Luciferase 17- |
| | mer, p21-Luciferase or MDM2-luciferase and |
| | with the empty plasmid pCDNA3 (invitrogen), |
| | which carries the neomycin resistant gene. |
| H1299 iASPP-V5/ | Human lung carcinoma stably transfected with |
| H1299 V5-iASPP | iASPP V5 tagged either at its N-terminal |
| | (V5-iASPP) or at its C-terminal (iASPP-V5). |

Maintaining Cell Lines

All the cell lines were cultured in DMEM from Gibco supplemented with L-Glutamine (2 mM, Gibco), penicillin/streptomycin (200 units/ml, Gibco) and 10% (v/v) of foetal calf serum) in flasks or dishes (Falcon) maintained in Heraecus incubators at 37° C. in the presence of 10% CO₂. Medium was changed every 3-5 days depending on the cell lines. On reaching confluence, the cells were washed once with PBS and incubated with 2-4 ml pre-warmed Trypsin-EDTA (Gibco, BRL) at 37° C. until the cells detached from the flasks or dishes. Trypsin was inhibited by addition of an appropriate volume of fresh growth medium and this culture was then seeded on to fresh flasks or dishes at the desired density.

Western Blotting

Cell lysates in sample buffer were loaded on to SDS polyacrylamide gels and the proteins separated at a constant voltage of 80 V. A protein molecular weight marker (Prestained Protein Marker Broad Range, Biolabs) was loaded and equal amounts of protein were loaded in each lane as determined by the BioRad assay system, unless otherwise stated. After the samples were separated through the gel, the gel was transferred to a wet transfer unit and the proteins blotted onto nitro-cellulose membrane (Schleicher and Schull, Germany) for 3 hrs at a constant voltage of 55 V or 20V overnight in a Hoefer Transphor Electrophoresis unit. The membrane was then stained with Ponceau S solution to determine the success of the transfer of proteins and equal loading of the lanes. The membranes were then washed in water and incubated in 10% non-fat milk at room temperature for 40-60 minutes. The membranes were then ready to be probed with primary antibody at the recommended concentrations for 1-3 hr at room temperature or overnight at 4° C. The blots were washed with large amounts of water before addition of the secondary HRP-conjugated antibody at the recommended concentration (generally 1:2000) at room temperature for 1 hr. After incubation with the secondary antibody the membrane was washed with 1×TBS-T (10 mM Tris pH 8.0, 150 mM NaCl, 0.5% Tween 20) extensively with repeated changes of TBS-T. The ECL was then performed according the manufacturer's instructions (Amersham Life Science, UK). The membrane was covered with Saran Wrap™ and exposed to Hyperfilm™ (Amersham Life Science, UK) for varying lengths of time to obtain an optimal exposure. If reprobing with another primary antibody was required, stripping of blots was performed. Blots were incubated with stripping buffer and freshly added mercaptoethanol in a flat bottomed tray at 55° C. on a shaker for 30 minutes or with a commercial stripping buffer (Chemicon Internaional, USA) for 15 minutes at room temperature. The blots were extensively washed with TBST and then blocked in 10% milk for 1 hour at room temperature. The blot was then reprobed with primary antibody as before.

Cell-Based Assays

Construction of Stable Cell Lines

Cells were co-transfected with 1 μg of the plasmid of interest and 4 μg of pCDNA3, which contains the resistance gene against neomycin for 6 hours. They were washed once with PBS and cultured in medium supplemented with neomycin. Twenty four hours later they were split and plated at different dilutions (from 1:50 to 1:200) in medium containing neomycin. The medium was changed every three days for two weeks until colonies formed. Colonies were picked and sub-cultured into a 24-well plate. After proliferation of each colony, cells were characterized either by luciferase assays or by western blotting according to the cell line.

Cell Transfection

Adherent cells were grown to a confluence of 70-80% in fresh medium. Cells were plated 24 hours prior to the transfection procedure. The medium on the dishes was replaced with 3 ml of fresh medium 15-30 minutes before the transfection. 2×HBS buffer (280 mM NaCl, 10 mM KCL, 1.4 mM Na₂HPO₄.2H₂O, 12 mM glucose, 39 mM HEPES, adjusted to a pH 6.9-7.3) was diluted in sterile water to a concentration of 1× in a final volume of 300 μl. The required amount of DNA was added using sterile Gilson tips and mixed thoroughly. To form the precipitate, 12.5 μl of 2.5 M CaCl₂ was added to the transfection mix and left at room temperature for 12-15 minutes. Six hours later dishes were washed with medium without serum once before adding 3 ml of fresh medium supplemented with 10% serum until further analysis of cells.

Transactivation Assays

Twenty four hours after transfection, the cells were lysed in Reporter Lysis buffer supplied by Promega and assayed using the Luciferase assay kit (Promega), measuring luciferase activity with the Autolumat Plus (Berthold technologies). The fold activation of a particular reporter was determined by the activity of the transfected plasmid divided by the activity of vector alone. The fold increase of activity in response to drug treatment was obtained by the activity in the presence of drug divided by the activity of the untreated sample.

### EXAMPLE 1

### Common Chemotherapy Drugs Differ in their P53-Dependent Transactivation Activity on Proapoptotic Promoter Pig3

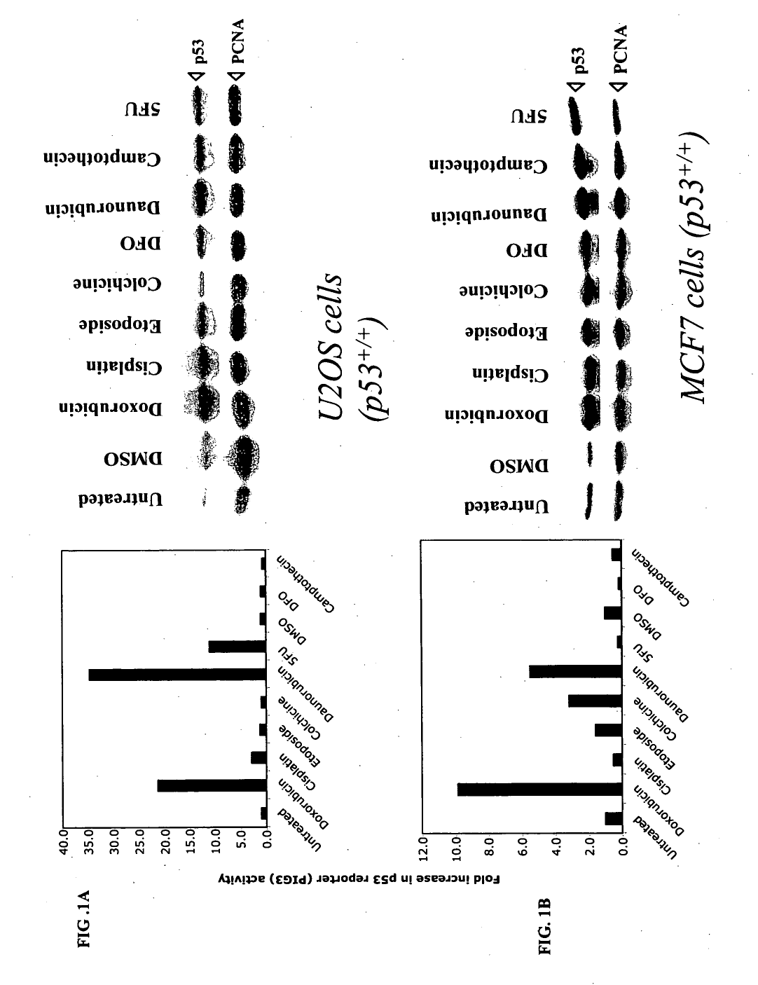

Some of the drugs commonly used in cancer therapy are known to stimulate p53 transactivation activity. To test their effect on a proapoptotic promoter specifically transactivated by p53, U2OS and MCF7 cells (both wild type p53) were transiently transfected with the proapoptotic reporter Pig3Luciferase and then treated for 24 hours with eight different compounds (FIGS. 1A and 1B). In both cell lines, the ability of p53 to transactivate the Pig3 promoter was strongly increased by doxorubicin and daunorubicin treatments. 5FU is also able to induce such induction in U2OS. By examining at the p53 level on western blots, a discordance was revealed between p53 level and its ability to transactivate the Pig3 promoter in response to different chemotherapy drugs. p53 was activated by addition of drugs to the medium (difference in level between control and all the drugs) but the activation of the Pig3 promoter in response to drugs did not directly correlate with the p53 protein level. For example, cisplatin and daunorubicin induced similar level of p53 but Pig3 promoter activity went up by 2.9 versus 34.7 fold respectively in U2OS.

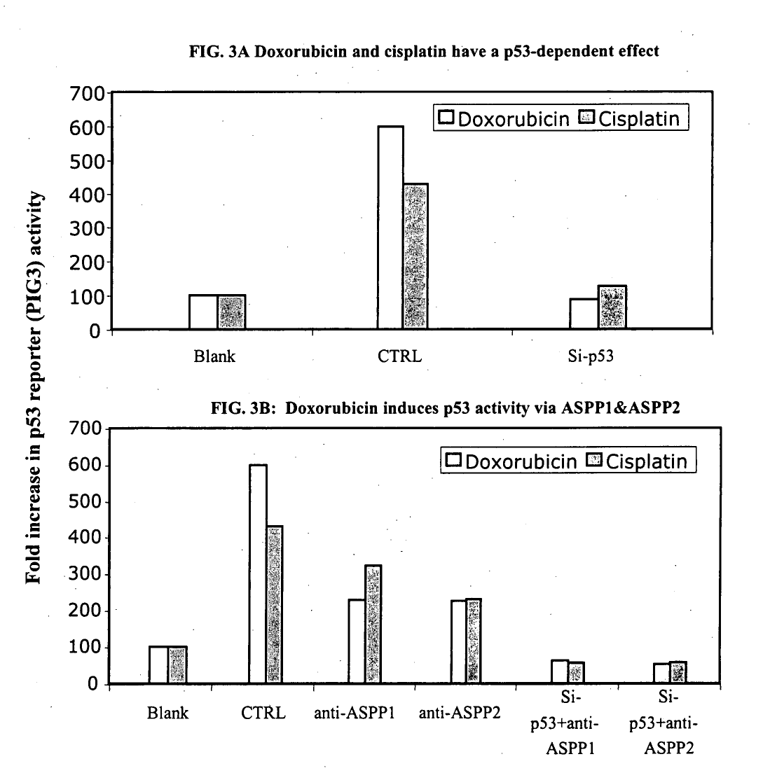

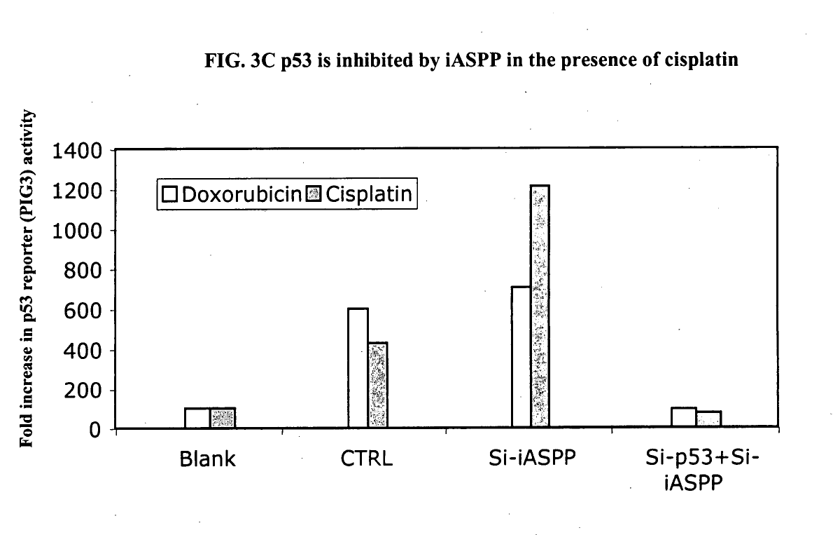

To demonstrate the role of p53 in the response to chemotherapy drugs, the short interfering RNA (Si—RNA) (Elbashir et al., 2001) technique was used to knock down p53. Si—RNA cloned into pSuper plasmid (Brummelkamp et al., Science. 296(5567):550-3, 2002) was transiently transfected with the Pig3 reporter into U2OS cells treated with doxorubicin or cisplatin (FIG. 3A). In the absence of p53 Si—RNA, p53 transactivated the Pig3 promoter as before but its presence completely abolished the ability of p53 to transactivate Pig3 with either drug. In this experiment, cisplatin was clearly shown to be involved in p53 activation but did not have the same efficiency as doxorubicin to stimulate proapoptotic promoter.

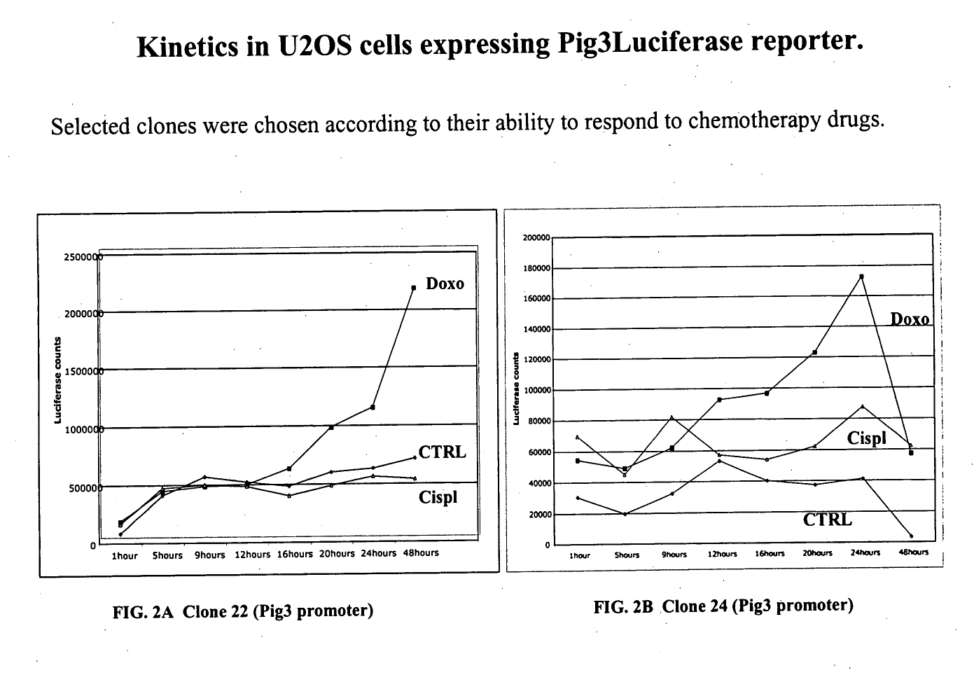

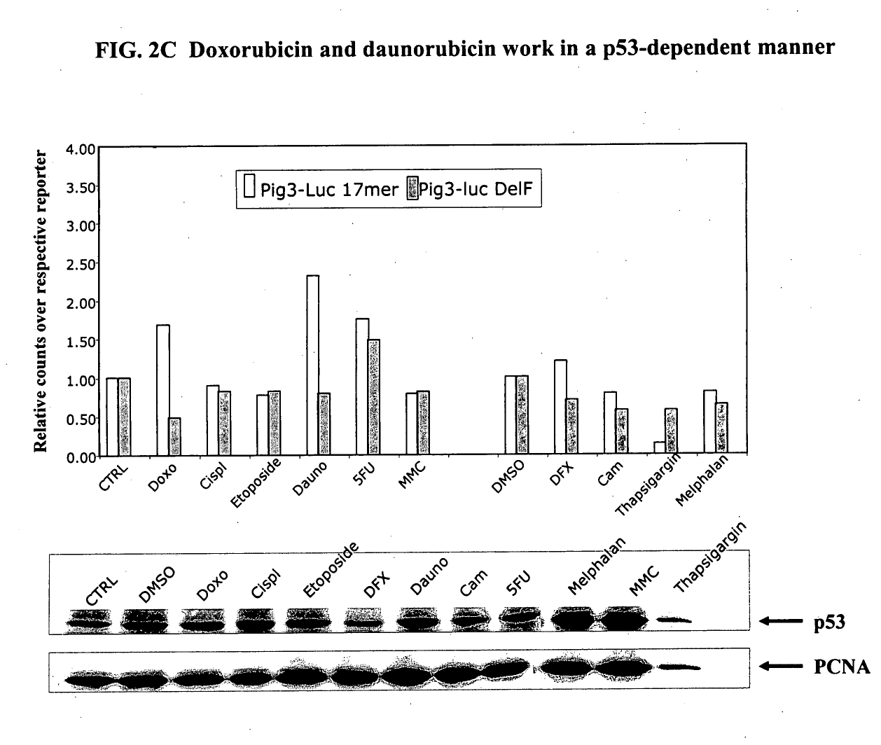

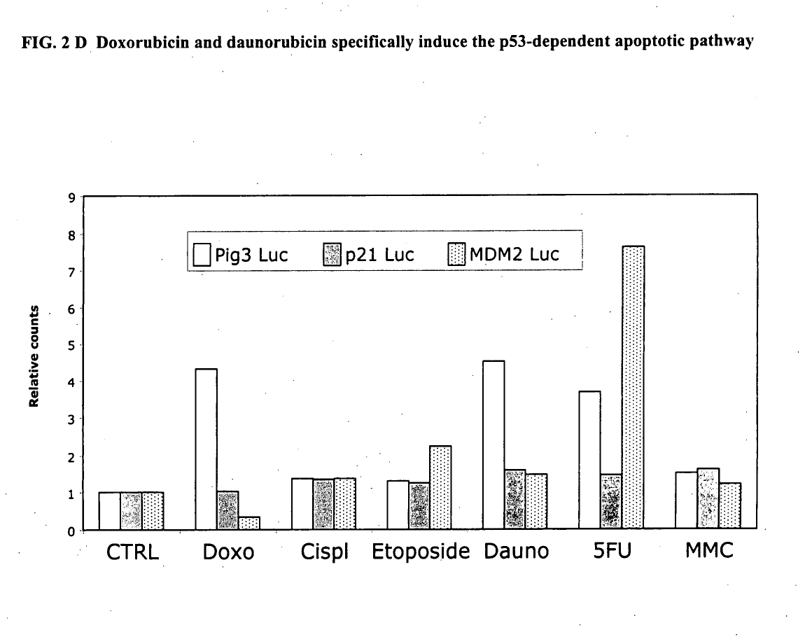

To extend these observations, stable cell lines were constructed to overcome the variations caused by transfection. Pig3Luciferase, Pig3-delFLuciferase (similar to the Pig3 promoter but without the p53 binding site), p21Luciferase, MDM2Luciferase reporters were used to generate four cell lines in U2OS cells. After selection, isolated clones were picked and checked for their ability to respond to drug treatments as shown in FIGS. 2A and 2B for two different Pig3Luciferase clones, which were shown to respond to doxorubicin. Six chemotherapy drugs were chosen for further study and they were tested for their ability to induce p53 responsive promoters. Comparing the Pig3 promoter with its deleted counterpart confirmed the observation seen with transient transfections (FIG. 2C). The p53 level could be the same but its ability to transactivate Pig3 promoter was different depending on the drug. Doxorubicin and daunorubicin did not affect the activity of the deleted Pig3 promoter indicating a p53-dependent effect for these two drugs. The absence or the slight reduction observed with the others drugs did not necessary mean that p53 did not play a role in cellular response under these treatments.

Additional cell lines containing the p21 or MDM2 promoters linked to luciferase were also tested (FIG. 2D). Doxorubicin and daunorubicin specifically stimulated the Pig3-Luciferase whereas cisplatin, etoposide and mitomycin C did not affect the activity of any promoters. It is noteworthy that 5-Fluorouracil induced the Pig3 promoter activity by three fold but also had a significant effect (7 fold) on the MDM2 promoter.

From these data we can classify some of the chemotherapy drugs used in this study into two groups for the p53 response: doxorubicin-like drugs and cisplatin-like drugs, which were both able to stabilize p53 but were not able to induce the same intensity of response on the proapototic promoter Pig3. As the p53 level alone could not explain the degree of transactivation of the Pig3 gene, p53 co-factors might have an important role in response to certain drugs. The ASPP proteins family known to specifically stimulate the apoptotic activity of p53 would be good candidates to investigate in this context.

### EXAMPLE 2

### Doxorubicin-Like Drugs Specifically Enhance ASPP1 and ASPP2 Responses

To understand if ASPP family proteins are able to influence the p53 response to chemotherapy drugs, antisense silencing of ASPP1 and ASPP2 (FIG. 3B) was tested in the presence or absence of p53 Si—RNA. The introduction of the ASPP1 and ASPP2 antisense significantly reduced the activity of the Pig3 reporter in presence of doxorubicin but caused only a small reduction of the cisplatin induction of the Pig3 promoter. The co-transfection of these antisense RNA with the p53 Si—RNA greatly reduced the promoter activity to a level lower than the blank.

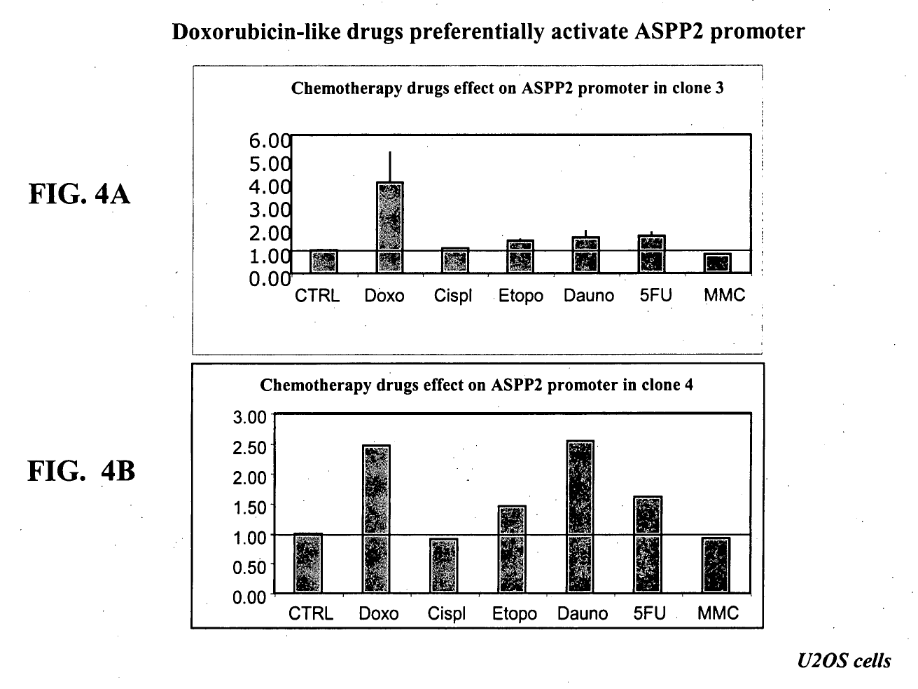

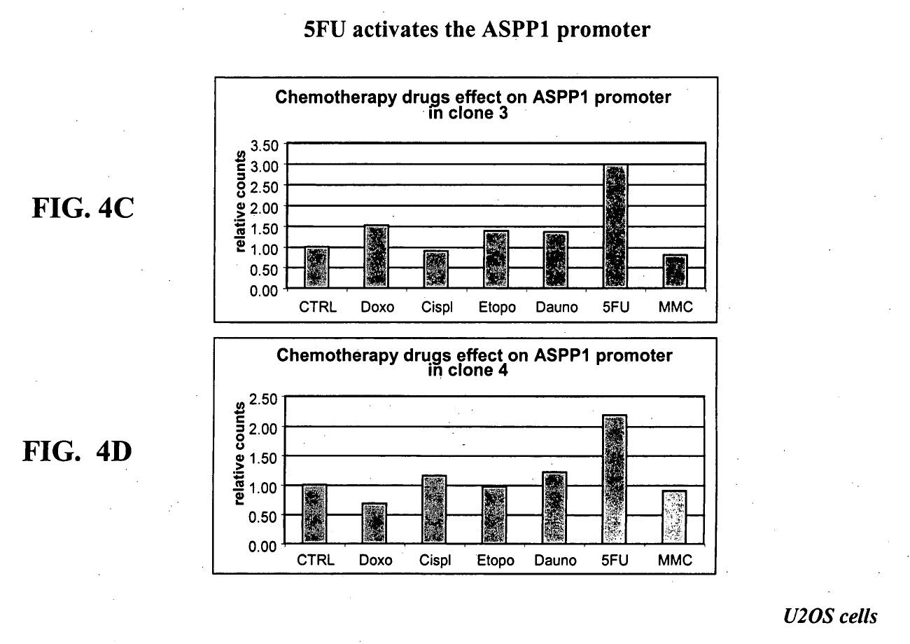

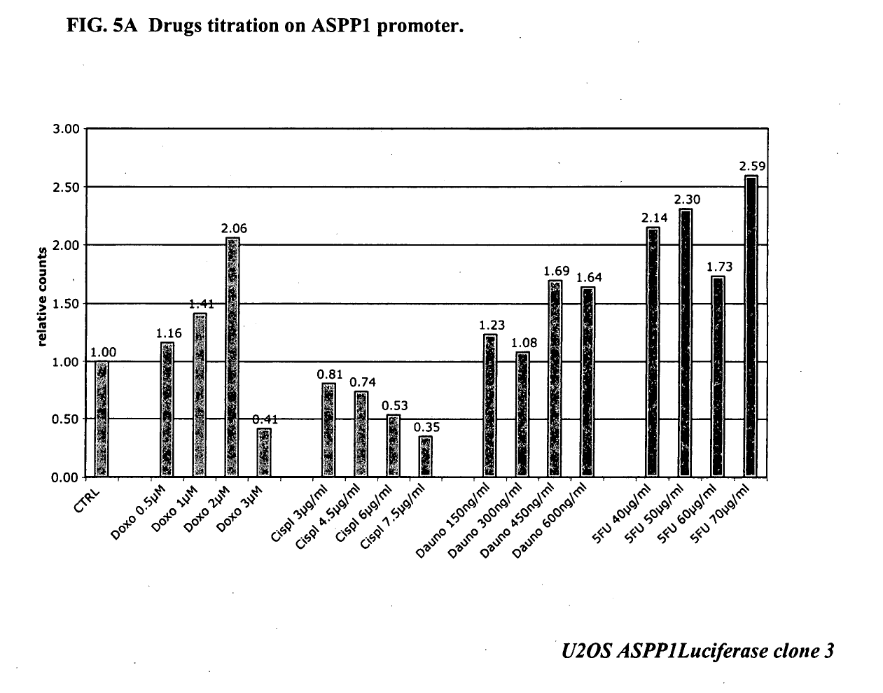

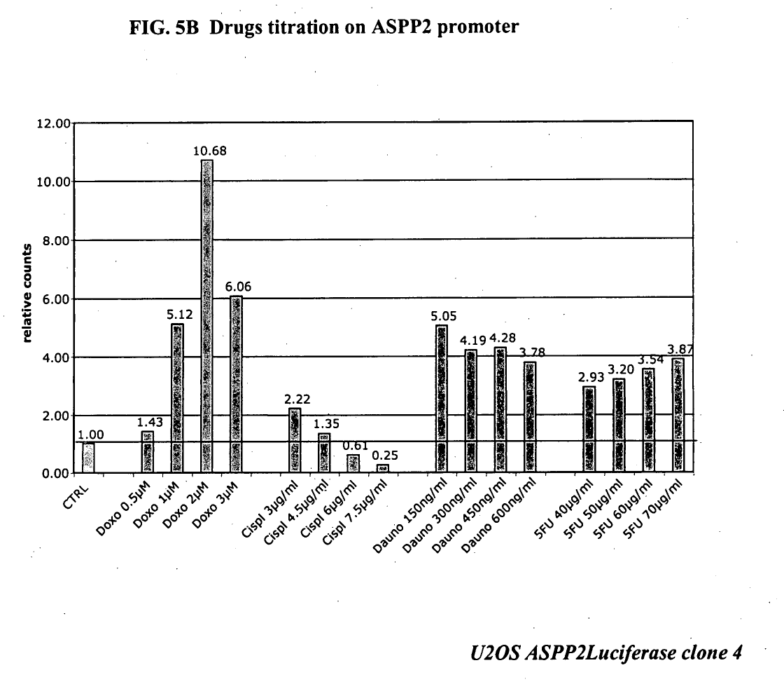

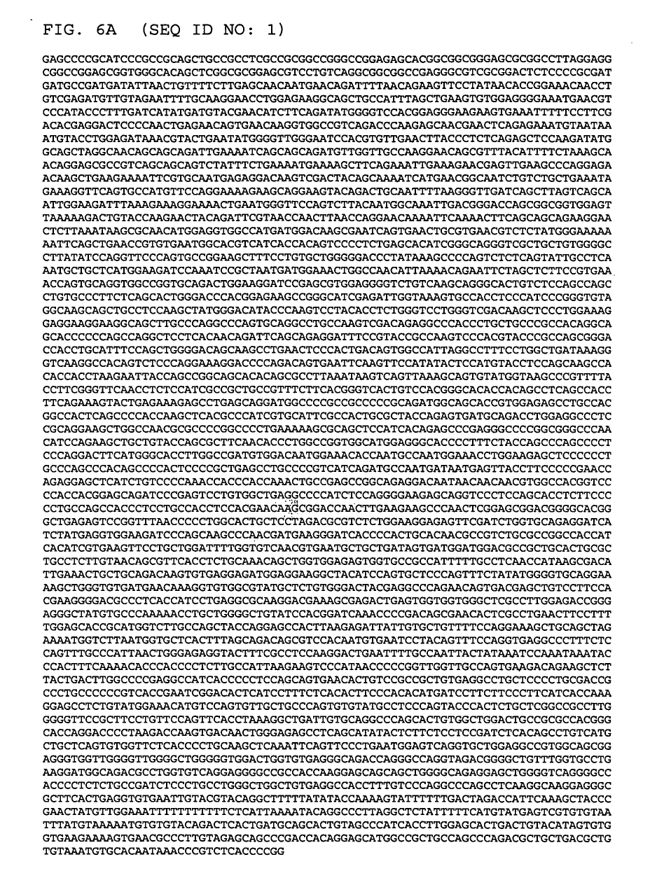

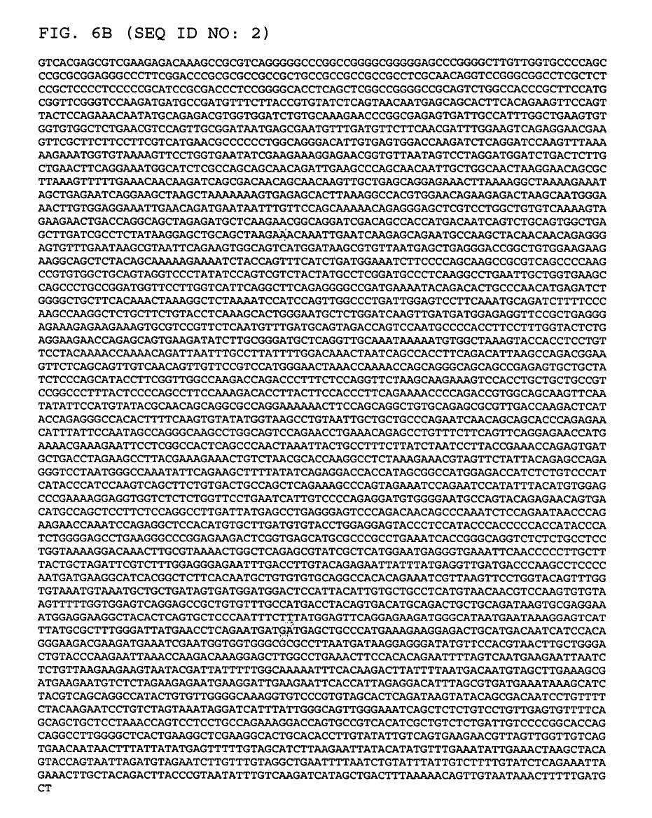

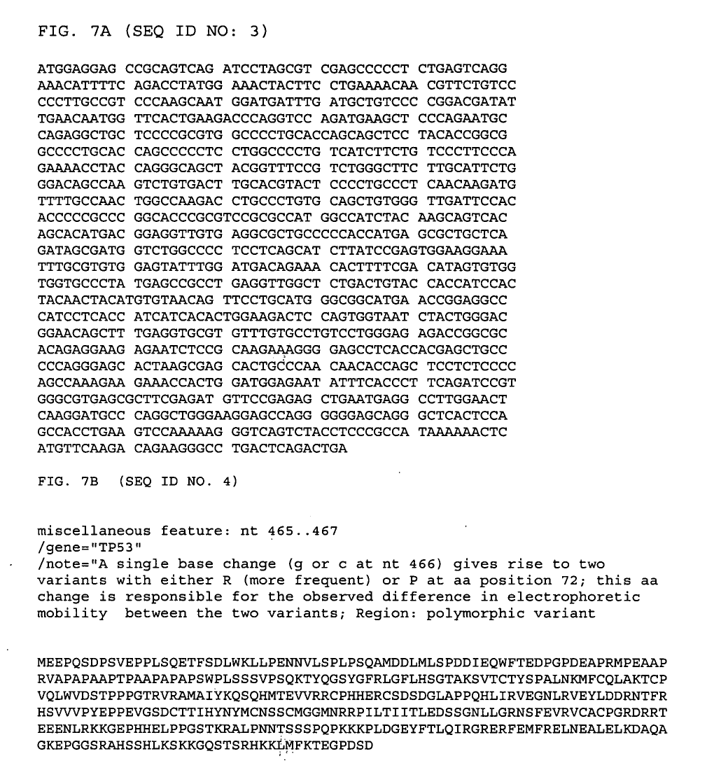

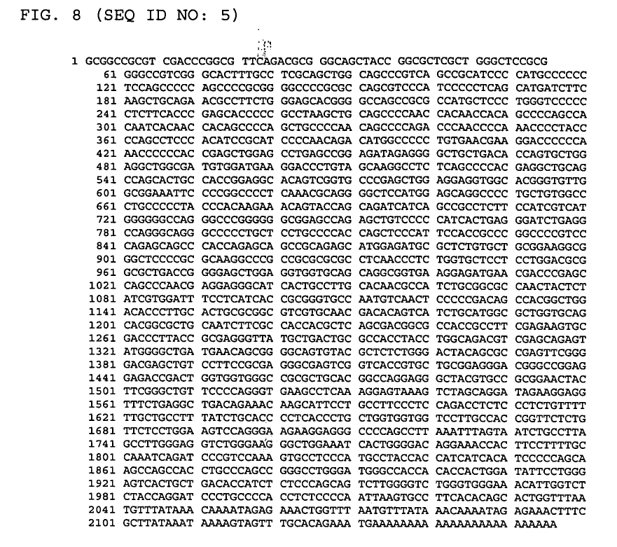

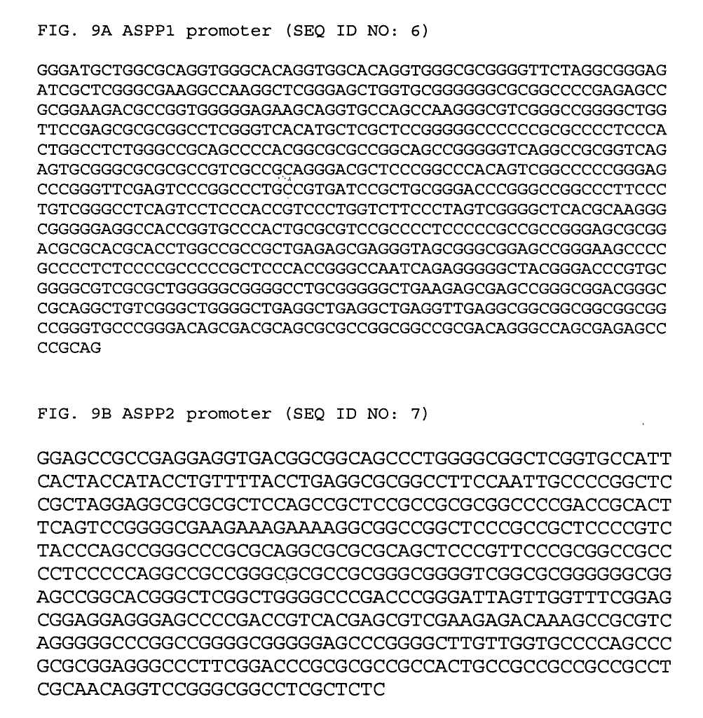

ASPP might respond to drugs in many ways in cells. To start investigating this, stable cell lines expressing luciferase under the control of the ASPP1 or ASPP2 promoters were created in U2OS cells. For each cell line, two clones were tested with the six drugs used previously. With the ASPP1 promoter, a significant effect was observed with 5-Fluorouracil after 24 hours (FIGS. 4C and D). The other drugs did not seem to stimulate the activity of the promoter. With the ASPP2 promoter (FIG. 4A), results were not completely similar in the two clones used. In clone 4, the promoter was stimulated by the addition of doxorubicin and daunorubicin (FIG. 4B) whereas in the clone 3 only doxorubicin had an effect on the activity (FIG. 4A). At the same time, these cell lines were used to titrate drugs. As little as 40 μg/ml of 5FU can induce the maximum ASPP1Luciferase reporter activity (FIG. 5A). Doxorubicin had a moderate effect (2 fold) at 2 μM but appeared to be toxic if the concentration reached 3 μM. Cisplatin and daunorubicin had little effect regardless of the concentration. With the ASPP2 promoter activity using the clone 4 (FIG. 5B), doxorubicin had its highest effect at 2 μM (10 fold increase over the control) and daunorubicin increased the activity by 4 fold at 150 ng/ml. Again cisplatin had no effect and the 5-Fluorouracil started to activate the promoter at 40 μg/ml by 3 fold.

Taken together, these results suggested that doxorubicin-like drugs stimulated the activity of p53 by enhancing the activity of ASPP1 and ASPP2. This could be partly achieved by enhancing the expression of ASPP1 and ASPP2.

### EXAMPLE 3

### Cisplatin-Like Drugs Fail to Remove the Inhibitory Effect of iASPP on p₅₃

In contrast to results obtained with ASPP1 and ASPP2, knockdown of iASPP with an Si—RNA stimulated the activity of the Pig3 promoter by three fold with cisplatin treatment (FIG. 3C). Again co-expression of p53 Si—RNA reduced Activity to background levels. These data indicate that endogenous p53 activity was induced by doxorubicin cooperating with ASPP1 and ASPP2 in U2OS cells but in response to cisplatin endogenous p53 activity was being restrained by iASPP.

### EXAMPLE 4

### ASPP Proteins are Involved in the p53-Apoptotic Response to Some of the Chemotherapy Drugs

The data show that p53 is able to increase the activity of the Pig3 promoter when U2OS and MCF7 cells are treated with doxorubicin, daunorubicin and 5FU compared to the other drugs, even though these drugs are well characterized to act through a p53-dependent mechanism in the literature. By using stable cell lines expressing luciferase reporters we have shown that doxorubicin and daunorubicin specifically stimulate the Pig3 promoter and not p21 or MDM2, indicating that these two drugs preferentially stimulate the apoptotic pathway of p53 rather than the cell cycle arrest. The doxorubicin effect on apoptosis is highlighted by the fact that in presence of antisense ASPP 1 and antisense ASPP2, the drug became less effective whereas there was no effect with cisplatin. Measuring the activity of ASPP1 and ASPP2 promoters by luciferase assays revealed 5FU to be a good activator of the ASPP1 promoter and the two DNA intercalators (doxorubicin and daunorubicin) to preferentially activate the ASPP2 promoter. The discrepancy observed between the two clones expressing the ASPP2Luciferase reporter will be addressed by using a pool of stably transfected cells. However, cisplatin does not show any difference in the activity of the promoters and it does not increase the p53 transactivation ability on proapoptotic promoter such as Pig3.

To fully understand the role played by ASPP1 and ASPP2 in response to chemotherapy drugs, it is important to look at the activity of their promoters at shorter time than 24 hours to appreciate how reactive they are under stress conditions. Initial observations at the protein level did not show a significant variation at 24 hours.

1. A method to diagnose and treat an animal having a condition which would benefit from stimulation of apoptosis, comprising:
determining an expression pattern of at least one nucleic acid molecule in the animal, wherein decreased expression of the at least one nucleic acid molecule indicates that the subject would benefit from administration of a chemotherapeutic drug; and
administering at least one chemotherapeutic agent which stimulates expression of the at least one nucleic acid molecule, wherein the at least one nucleic acid comprises:
a) a nucleic acid molecule comprising the nucleic acid sequence shown in SEQ ID NO: 1 or 2;
b) a nucleic acid molecule which hybridizes under stringent hybridization conditions to the nucleic acid molecule shown in SEQ ID NO: 1 or 2, wherein the nucleic acid molecule which hybridizes encodes a polypeptide which stimulates the apoptotic activity of p53;
c) a nucleic acid molecule comprising a nucleic acid sequence which is degenerate as a result of the genetic code to the nucleic acid molecule of (a) or (b).

2. The method of claim 1, wherein determining an expression pattern of at least one nucleic acid molecule is determined in a cell/tissue sample obtained from the animal.

3. The method of claim 1, wherein the cell/tissue sample comprises a cancer cell.

4. The method of claim 1, wherein the cell/tissue sample is a breast cell/tissue sample.

5. The method of claim 1, further comprising determining a p53 genotype of the animal.

6. The method of claim 5, wherein the p53 genotype is a p53 variant comprising a substitution of an amino acid residue encoded by codon 72 of the nucleic acid sequence shown in SEQ ID NO: 3.

7. The method of claim 6, wherein codon 72 encodes a praline or an arginine.

8. The method of claim 1, further comprising determining the expression of a nucleic acid molecule, wherein the nucleic acid molecule comprises:
i) a nucleic acid molecule comprising the nucleic acid sequence shown in SEQ ID NO: 5;
ii) a nucleic acid molecule which hybridizes under stringent hybridization conditions to a nucleic acid molecule comprising SEQ ID NO: 5 and which encodes a polypeptide which inhibits the apoptotic activity of p53;
iii) a nucleic acid molecule comprising a nucleic acid sequence which is degenerate as a result of the genetic code to the nucleic acid molecule of (i) or (ii).

9. The method of claim 1, wherein the chemotherapeutic agent is a DNA intercalating agent or a topoisomerase inhibitor.

10. The method of claim 1, wherein the chemotherapeutic agent is an anthracyline antibiotic.

11. The method of claim 1, wherein the chemotherapeutic agent is doxorubicin, daunorubicin, or variant thereof.

12. The method of claim 1, wherein the chemotherapeutic agent is an anti-metabolic drug.

13. The method of claim 12, wherein the anti-metabolic drug is 5-fluorouracil and optionally includes leucovorin.

14. The method of claim 1, further comprising administering to the animal an antagonistic agent which inhibits the activity of a polypeptide encoded by a nucleic acid molecule, wherein the nucleic acid molecule comprises:
i) a nucleic acid molecule comprising a nucleic acid sequence as represented in SEQ ID NO: 5;
ii) a nucleic acid molecule which hybridises under stringent hybridisation conditions to a nucleic acid molecule comprising SEQ ID NO: 5 and which encodes a polypeptide which inhibits the apoptotic activity of p53;
iii) a nucleic acid molecule comprising a nucleic acid sequence which is degenerate as a result of the genetic code to the nucleic acid molecule of (i) or (ii).

15. The method of claim 14, wherein the antagonistic agent is an antibody, or active binding fragment thereof, which binds and inhibits the activity of said polypeptide.

16. The method of claim 15 wherein the antibody or active binding fragment thereof is a monoclonal antibody.

17. The method of claim 15, wherein the active binding fragment is a single chain antibody variable region fragment.

18. The method of claim 15, wherein the antibody is a humanised or a chimeric antibody.

19. The method of claim 14, wherein the antagonistic agent is an antisense nucleic acid molecule or RNAi molecule of the nucleic acid sequence shown in SEQ ID NO: 5.

20. The method of claim 19, wherein the antisense nucleic acid molecule or RNAi molecule is part of a vector.

21. The method of claim 14, wherein the antagonist agent comprises at least one platinum containing anti-cancer containing compound.

22. The method of claim 21, wherein said platinum containing compound is cisplatin; carboplatin; or oxaloplatin.

23. The method of claim 1, wherein the animal is a human.

24. The method of claim 1, further comprising administering p53 to the animal.

25. The method of claim 24, wherein the p53 is administered comprises the nucleic acid sequence shown in SEQ ID NO: 3 or a nucleic acid molecule which hybridizes to the nucleic acid sequence in SEQ ID NO: 3 and encodes a polypeptide with specific activity associated with p53.

26. The method of claim 24, wherein the p53 administered comprises a polymorphic variant modified at codon 72.

27. The method of claim 26, wherein the variant modified at codon 72 comprises p53Arg72 or p53Pro72.

28. The method of claim 25, wherein said nucleic acid molecule is part of a vector adapted for expression of at least p53.

29. A screening method to identify a chemotherapeutic agent which stimulates the expression of a nucleic acid molecule, comprising:
providing a preparation comprising at least one candidate agent to be tested and a vector adapted for expression of a detectable reporter molecule, wherein the expression of the detectable reporter molecule is controlled by a promoter sequence which naturally controls the expression of the nucleic acid molecule; and
determining the expression of the detectable reporter molecule in the presence of the candidate agent, wherein the nucleic acid molecule comprises:
a) the nucleic acid sequence shown in SEQ ID NO: 1 or 2;
b) a nucleic acid molecule which hybridizes under stringent hybridization conditions to SEQ ID NO: 1 or 2 and which encodes a polypeptide which stimulates the apoptotic activity of p53;
c) a nucleic acid molecule comprising a nucleic acid sequence which is degenerate as a result of the genetic code to the nucleic acid molecule of (a) or (b).

30. The method of claim 29, wherein the preparation comprises a cell transfected with the vector.

31. The method of claim 30, wherein said cell is a eukaryotic mammalian cell.

32. The method of claim 30, wherein the cell is a cancer cell.

33. The method of claim 29, wherein the promoter sequence comprises the nucleic acid sequence shown in SEQ ID NO: 6 or 7 or a variant sequence of SEQ ID NO: 6 or 7, wherein the variant sequence has been modified by addition, deletion or substitution of at least one nucleotide base, wherein the variant sequence substantially retains the transcriptional activity of the promoter sequence shown in SEQ ID NO: 6 or 7 or has enhanced activity when compared to the unmodified promoter sequence.

34. The method of claim 29, wherein the candidate agent is a DNA intercalating drug or topoisomerase inhibitor.

35. The method of claim 29, wherein the candidate agent is an anti-metabolic drug.

36. A vector comprising a promoter sequence, wherein the promoter sequence comprises the nucleic acid molecule shown in SEQ ID NO: 6 or 7 or comprises a variant sequence of SEQ ID NO: 6 or 7, wherein the variant sequence has been modified by addition, deletion or substitution of at least one nucleotide base, and wherein the variant sequence substantially retains the transcriptional activity of the promoter sequence shown in SEQ ID NO: 6 or 7, or has enhanced activity when compared to the unmodified promoter sequence.

37. The vector of claim 36, wherein the promoter sequence is operably linked to a reporter molecule.

38. A non-human transgenic animal comprising the vector of claim 36.

39. The non-human transgenic animal of claim 38, wherein the animal is a rodent.

40. The non-human transgenic animal of claim 39, wherein the rodent is a mouse or rat.


SEQUENCE LISTING (7 sequences)

SEQ ID NO: 1 - DNA, 4834 bp, Homo sapiens
gagccccgcatcccgccgcagctgccgcctcgccgcggccgggccggagagcacggcggcgggagcgcggccttaggaggcggccggagcggtgggcacagctcggcgcggagcgtcctgtcaggcggcggccgagggcgtcgcggactctccccgcgatgatgccgatgatattaactgttttcttgagcaacaatgaacagattttaacagaagttcctataacaccggaaacaacctgtcgagatgttgtagaattttgcaaggaacctggagaaggcagctgccatttagctgaagtgtggaggggaaatgaacgtcccataccctttgatcatatgatgtacgaacatcttcagatatggggtccacggagggaagaagtgaaatttttccttcgacacgaggactccccaactgagaacagtgaacaaggtggccgtcagacccaagagcaacgaactcagagaaatgtaataaatgtacctggagataaacgtactgaatatggggttgggaatccacgtgttgaacttaccctctcagagctccaagatatggcagctaggcaacagcagcagattgaaaatcagcagcagatgttggttgccaaggaacagcgtttacattttctaaagcaacaggagcgccgtcagcagcagtctatttctgaaaatgaaaagcttcagaaattgaaagaacgagttgaagcccaggagaacaagctgaagaaaattcgtgcaatgagaggacaagtcgactacagcaaaatcatgaacggcaatctgtctgctgaaatagaaaggttcagtgccatgttccaggaaaagaagcaggaagtacagactgcaattttaagggttgatcagcttagtcagcaattggaagatttaaagaaaggaaaactgaatgggttccagtcttacaatggcaaattgacgggaccagcggcggtggagttaaaaagactgtaccaagaactacagattcgtaaccaacttaaccaggaacaaaattcaaaacttcagcagcagaaggaactcttaaataagcgcaacatggaggtggccatgatggacaagcgaatcagtgaactgcgtgaacgtctctatgggaaaaaaattcagctgaaccgtgtgaatggcacgtcatcaccacagtcccctctgagcacatcgggcagggtcgctgctgtggggccttatatccaggttcccagtgccggaagctttcctgtgctgggggaccctataaagccccagtctctcagtattgcctcaaatgctgctcatggaagatccaaatccgctaatgatggaaactggccaacattaaaacagaattctagctcttccgtgaaaccagtgcaggtggccggtgcagactggaaggatccgagcgtggaggggtctgtcaagcagggcactgtctccagccagcctgtgcccttctcagcactgggacccacggagaagccgggcatcgagattggtaaagtgccacctcccatcccgggtgtaggcaagcagctgcctccaagctatgggacatacccaagtcctacacctctgggtcctgggtcgacaagctccctggaaaggaggaaggaaggcagcttgcccaggcccagtgcaggcctgccaagtcgacagaggcccaccctgctgcccgccacaggcagcaccccccagccaggctcctcacaacagattcagcagaggatttccgtaccgccaagtcccacgtacccgccagcgggaccacctgcatttccagctggggacagcaagcctgaactcccactgacagtggccattaggcctttcctggctgataaagggtcaaggccacagtctcccaggaaaggaccccagacagtgaattcaagttccatatactccatgtacctccagcaagccacaccacctaagaattaccagccggcagcacacagcgccttaaataagtcagttaaagcagtgtatggtaagcccgttttaccttcgggttcaacctctccatcgccgctgccgtttcttcacgggtcactgtccacgggcacaccacagcctcagccaccttcagaaagtactgagaaagagcctgagcaggatggccccgccgcccccgcagatggcagcaccgtggagagcctgccacggccactcagccccaccaagctcacgcccatcgtgcattcgccactgcgctaccagagtgatgcagacctggaggccctccgcaggaagctggccaacgcgccccggcccctgaaaaagcgcagctccatcacagagcccgagggccccggcgggcccaacatccagaagctgctgtaccagcgcttcaacaccctggccggtggcatggagggcacccctttctaccagcccagcccctcccaggacttcatgggcaccttggccgatgtggacaatggaaacaccaatgccaatggaaacctggaagagctcccccctgcccagcccacagccccactccccgctgagcctgccccgtcatcagatgccaatgataatgagttaccttcccccgaaccagaggagctcatctgtccccaaaccacccaccaaactgccgagccggcagaggacaataacaacaacgtggccacggtccccaccacggagcagatcccgagtcctgtggctgaggccccatctccaggggaagagcaggtccctccagcacctcttccccctgccagccaccctcctgccacctccacgaacaagcggaccaacttgaagaagcccaactcggagcggacggggcacgggctgagagtccggtttaaccccctggcactgctcctagacgcgtctctggaaggagagttcgatctggtgcagaggatcatctatgaggtggaagatcccagcaagcccaacgatgaagggatcaccccactgcacaacgccgtctgcgccggccaccatcacatcgtgaagttcctgctggattttggtgtcaacgtgaatgctgctgatagtgatggatggacgccgctgcactgcgctgcctcttgtaacagcgttcacctctgcaaacagctggtggagagtggtgccgccatttttgcctcaaccataagcgacattgaaactgctgcagacaagtgtgaggagatggaggaaggctacatccagtgctcccagtttctatatggggtgcaggaaaagctgggtgtgatgaacaaaggtgtggcgtatgctctgtgggactacgaggcccagaacagtgacgagctgtccttccacgaaggggacgccctcaccatcctgaggcgcaaggacgaaagcgagactgagtggtggtgggctcgccttggagaccgggagggctatgtgcccaaaaacctgctggggctgtatccacggatcaaaccccgacagcgaacactcgcctgaacttccttttggagcaccgcatggtcttgccagctaccaggagccacttaagagattattgtgctgttttccaggaaagctgcagctagaaaatggtcttaatggtgctcactttagcagacagcgtccacaatgtgaatcctacagtttccaggtgaggccctttctccagtttgcccattaactgggagaggtactttcgcctccaaggactgaattttgccaattactataaatccaaataaatacccactttcaaaacacccacccctcttgccattaagaagtcccataacccccggttggttgccagtgaagacagaagctcttactgacttggccccgaggccatcaccccctccagcagtgaacactgtccgccgctgtgaggcctgctcccctgcgaccgccctgccccccgtcaccgaatcggacactcatcctttctcacacttcccacacatgatccttcttcccttcatcaccaaaggagcctctgtatggaaacatgtccagtgttgctgcccagtgtgtatgcctcccagtacccactctgctcggccgccttgggggttccgcttcctgttccagttcacctaaaggctgattgtgcaggcccagcactgtggctggactgccgcgccacgggcaccaggacccctaagaccaagtgacaactgggagagcctcagcatatactcttctcctccgatctcacagcctgtcatgctgctcagtgtggttctcacccctgcaagctcaaattcagttccctgaatggagtcaggtgctggaggccgtggcagcggagggtggttggggttggggctgggggtggactggtgtgagggcagaccagggccaggtagacggggctgtttggtgcctgaaggatggcagacgcctggtgtcaggaggggccgccaccaaggagcagcagctggggcagaggagctggggtcaggggccacccctctctgccgatctccctgcctgggctggctgtgaggccacctttgtcccaggcccagcctcaaggcaaggagggcgcttcactgaggtgtgaattgtacgtacaggctttttatataccaaaagtattttttgactagaccattcaaagctacccgaactatgttggaaatttttttttttctcattaaaatacaggcccttaggctctatttttcatgtatgagtcgtgtgtaatttatgtaaaaatgtgtgtacagactcactgatgcagcactgtagcccatcaccttggagcactgactgtacatagtgtggtgaagaaaagtgaacgcccttgtagagcagcccgaccacaggagcatggccgctgccagcccagacgctgctgacgctgtgtaaatgtgcacaataaacccgtctcaccccgg

SEQ ID NO: 2 - DNA, 4402 bp, Homo sapiens
gtcacgagcgtcgaagagacaaagccgcgtcagggggcccggccggggcgggggagcccggggcttgttggtgccccagcccgcgcggagggcccttcggacccgcgcgccgccgctgccgccgccgccgcctcgcaacaggtccgggcggcctcgctctccgctcccctcccccgcatccgcgaccctccggggcacctcagctcggccggggccgcagtctggccacccgcttccatgcggttcgggtccaagatgatgccgatgtttcttaccgtgtatctcagtaacaatgagcagcacttcacagaagttccagttactccagaaacaatatgcagagacgtggtggatctgtgcaaagaacccggcgagagtgattgccatttggctgaagtgtggtgtggctctgaacgtccagttgcggataatgagcgaatgtttgatgttcttcaacgatttggaagtcagaggaacgaagttcgcttcttccttcgtcatgaacgcccccctggcagggacattgtgagtggaccaagatctcaggatccaagtttaaaaagaaatggtgtaaaagttcctggtgaatatcgaagaaaggagaacggtgttaatagtcctaggatggatctgactcttgctgaacttcaggaaatggcatctcgccagcagcaacagattgaagcccagcaacaattgctggcaactaaggaacagcgcttaaagtttttgaaacaacaagatcagcgacaacagcaacaagttgctgagcaggagaaacttaaaaggctaaaagaaatagctgagaatcaggaagctaagctaaaaaaagtgagagcacttaaaggccacgtggaacagaagagactaagcaatgggaaacttgtggaggaaattgaacagatgaataatttgttccagcaaaaacagagggagctcgtcctggctgtgtcaaaagtagaagaactgaccaggcagctagagatgctcaagaacggcaggatcgacagccaccatgacaatcagtctgcagtggctgagcttgatcgcctctataaggagctgcagctaagaaacaaattgaatcaagagcagaatgccaagctacaacaacagagggagtgtttgaataagcgtaattcagaagtggcagtcatggataagcgtgttaatgagctgagggaccggctgtggaagaagaaggcagctctacagcaaaaagaaaatctaccagtttcatctgatggaaatcttccccagcaagccgcgtcagccccaagccgtgtggctgcagtaggtccctatatccagtcgtctactatgcctcggatgccctcaaggcctgaattgctggtgaagccagccctgccggatggttccttggtcattcaggcttcagaggggccgatgaaaatacagacactgcccaacatgagatctggggctgcttcacaaactaaaggctctaaaatccatccagttggccctgattggagtccttcaaatgcagatcttttcccaagccaaggctctgcttctgtacctcaaagcactgggaatgctctggatcaagttgatgatggagaggttccgctgagggagaaagagaagaaagtgcgtccgttctcaatgtttgatgcagtagaccagtccaatgccccaccttcctttggtactctgaggaagaaccagagcagtgaagatatcttgcgggatgctcaggttgcaaataaaaatgtggctaaagtaccacctcctgttcctacaaaaccaaaacagattaatttgccttattttggacaaactaatcagccaccttcagacattaagccagacggaagttctcagcagttgtcaacagttgttccgtccatgggaactaaaccaaaaccagcagggcagcagccgagagtgctgctatctcccagcataccttcggttggccaagaccagaccctttctccaggttctaagcaagaaagtccacctgctgctgccgtccggccctttactccccagccttccaaagacaccttacttccacccttcagaaaaccccagaccgtggcagcaagttcaatatattccatgtatacgcaacagcaggcgccaggaaaaaacttccagcaggctgtgcagagcgcgttgaccaagactcataccagagggccacacttttcaagtgtatatggtaagcctgtaattgctgctgcccagaatcaacagcagcacccagagaacatttattccaatagccagggcaagcctggcagtccagaacctgaaacagagcctgtttcttcagttcaggagaaccatgaaaacgaaagaattcctcggccactcagcccaactaaattactgcctttcttatctaatccttaccgaaaccagagtgatgctgacctagaagccttacgaaagaaactgtctaacgcaccaaggcctctaaagaaacgtagttctattacagagccagagggtcctaatgggccaaatattcagaagcttttatatcagaggaccaccatagcggccatggagaccatctctgtcccatcatacccatccaagtcagcttctgtgactgccagctcagaaagcccagtagaaatccagaatccatatttacatgtggagcccgaaaaggaggtggtctctctggttcctgaatcattgtccccagaggatgtggggaatgccagtacagagaacagtgacatgccagctccttctccaggccttgattatgagcctgagggagtcccagacaacagcccaaatctccagaataacccagaagaaccaaatccagaggctccacatgtgcttgatgtgtacctggaggagtaccctccatacccacccccaccatacccatctggggagcctgaagggcccggagaagactcggtgagcatgcgcccgcctgaaatcaccgggcaggtctctctgcctcctggtaaaaggacaaacttgcgtaaaactggctcagagcgtatcgctcatggaatgagggtgaaattcaacccccttgctttactgctagattcgtctttggagggagaatttgaccttgtacagagaattatttatgaggttgatgacccaagcctccccaatgatgaaggcatcacggctcttcacaatgctgtgtgtgcaggccacacagaaatcgttaagttcctggtacagtttggtgtaaatgtaaatgctgctgatagtgatggatggactccattacattgtgctgcctcatgtaacaacgtccaagtgtgtaagtttttggtggagtcaggagccgctgtgtttgccatgacctacagtgacatgcagactgctgcagataagtgcgaggaaatggaggaaggctacactcagtgctcccaatttctttatggagttcaggagaagatgggcataatgaataaaggagtcatttatgcgctttgggattatgaacctcagaatgatgatgagctgcccatgaaagaaggagactgcatgacaatcatccacagggaagacgaagatgaaatcgaatggtggtgggcgcgccttaatgataaggagggatatgttccacgtaacttgctgggactgtacccaagaattaaaccaagacaaaggagcttggcctgaaacttccacacagaattttagtcaatgaagaattaatctctgttaagaagaagtaatacgattatttttggcaaaaatttcacaagacttattttaatgacaatgtagcttgaaagcgatgaagaatgtctctagaagagaatgaaggattgaagaattcaccattagaggacatttagcgtgatgaaataaagcatctacgtcagcaggccatactgtgttggggcaaaggtgtcccgtgtagcactcagataagtatacagcgacaatcctgttttctacaagaatcctgtctagtaaataggatcatttattgggcagttgggaaatcagctctctgtcctgttgagtgttttcagcagctgctcctaaaccagtcctcctgccagaaaggaccagtgccgtcacatcgctgtctctgattgtccccggcaccagcaggccttggggctcactgaaggctcgaaggcactgcacaccttgtatattgtcagtgaagaacgttagttggttgtcagtgaacaataactttattatatgagtttttgtagcatcttaagaattatacatatgtttgaaatattgaaactaagctacagtaccagtaattagatgtagaatcttgtttgtaggctgaattttaatctgtatttattgtcttttgtatctcagaaattagaaacttgctacagacttacccgtaatatttgtcaagatcatagctgactttaaaaacagttgtaataaactttttgatgct

SEQ ID NO: 3 - DNA, 1182 bp, Homo sapiens
atggaggagccgcagtcagatcctagcgtcgagccccctctgagtcaggaaacattttcagacctatggaaactacttcctgaaaacaacgttctgtcccccttgccgtcccaagcaatggatgatttgatgctgtccccggacgatattgaacaatggttcactgaagacccaggtccagatgaagctcccagaatgccagaggctgctccccgcgtggcccctgcaccagcagctcctacaccggcggcccctgcaccagccccctcctggcccctgtcatcttctgtcccttcccagaaaacctaccagggcagctacggtttccgtctgggcttcttgcattctgggacagccaagtctgtgacttgcacgtactcccctgccctcaacaagatgttttgccaactggccaagacctgccctgtgcagctgtgggttgattccacacccccgcccggcacccgcgtccgcgccatggccatctacaagcagtcacagcacatgacggaggttgtgaggcgctgcccccaccatgagcgctgctcagatagcgatggtctggcccctcctcagcatcttatccgagtggaaggaaatttgcgtgtggagtatttggatgacagaaacacttttcgacatagtgtggtggtgccctatgagccgcctgaggttggctctgactgtaccaccatccactacaactacatgtgtaacagttcctgcatgggcggcatgaaccggaggcccatcctcaccatcatcacactggaagactccagtggtaatctactgggacggaacagctttgaggtgcgtgtttgtgcctgtcctgggagagaccggcgcacagaggaagagaatctccgcaagaaaggggagcctcaccacgagctgcccccagggagcactaagcgagcactgcccaacaacaccagctcctctccccagccaaagaagaaaccactggatggagaatatttcacccttcagatccgtgggcgtgagcgcttcgagatgttccgagagctgaatgaggccttggaactcaaggatgcccaggctgggaaggagccaggggggagcagggctcactccagccacctgaagtccaaaaagggtcagtctacctcccgccataaaaaactcatgttcaagacagaagggcctgactcagactga

SEQ ID NO: 4 - protein, 393 aa, Homo sapiens [sequence omitted]

SEQ ID NO: 5 - DNA, 2156 bp, Homo sapiens
gcggccgcgtcgacccggcgttcagacgcgggcagctaccggcgctcgctgggctccgcggggccgtcgggcactttgcctcgcagctggcagcccgtcagccgcatccccatgcccccctccagcccccagccccgcggggccccgcgccagcgtcccatccccctcagcatgatcttcaagctgcagaacgccttctgggagcacggggccagccgcgccatgctccctgggtcccccctcttcacccgagcacccccgcctaagctgcagccccaaccacaaccacagccccagccacaatcacaaccacagccccagctgccccaacagccccagacccaaccccaaacccctaccccagcctcccacatccgcatccccaacagacatggccccctgtgaacgaaggaccccccaaaccccccaccgagctggagcctgagccggagatagaggggctgctgacaccagtgctggaggctggcgatgtggatgaaggaccctgtagcaaggcctctcagccccacgaggctgcagccagcactgccaccggaggcacagtcggtgcccgagctggaggaggtggcacgggtgttggcggaaattccccggcccctcaaacgcaggggctccatggagcaggcccctgctgtggccctgccccctacccacaagaaacagtaccagcagatcatcagccgcctcttccatcgtcatggggggccagggcccggggggcggagccagagctgtcccccatcactgagggatctgaggccagggcagggccccctgctcctgccccaccagctcccattccaccgcccggccccgtcccagagcagcccaccagagcagccgcagagcatggagatgcgctctgtgctgcggaaggcgggctccccgcgcaaggcccgccgcgcgcgcctcaaccctctggtgctcctcctggacgcggcgctgaccggggagctggaggtggtgcagcaggcggtgaaggagatgaacgacccgagccagcccaacgaggagggcatcactgccttgcacaacgccatctgcggcgccaactactctatcgtggatttcctcatcaccgcgggtgccaatgtcaactcccccgacagccacggctggacacccttgcactgcgcggcgtcgtgcaacgacacagtcatctgcatggcgctggtgcagcacggcgctgcaatcttcgccaccacgctcagcgacggcgccaccgccttcgagaagtgcgacccttaccgcgagggttatgctgactgcgccacctacctggcagacgtcgagcagagtatggggctgatgaacagcggggcagtgtacgctctctgggactacagcgccgagttcggggacgagctgtccttccgcgagggcgagtcggtcaccgtgctgcggagggacgggccggaggagaccgactggtggtgggccgcgctgcacggccaggagggctacgtgccgcggaactacttcgggctgttccccagggtgaagcctcaaaggagtaaagtctagcaggatagaaggaggtttctgaggctgacagaaacaagcattcctgccttccctccagacctctccctctgttttttgctgcctttatctgcacccctcaccctgctggtggtggtccttgccaccggttctctgttctcctggaagtccagggaagaaggagggccccagccttaaatttagtaatctgccttagccttgggaggtctgggaagggctggaaatcactggggacaggaaaccacttccttttgccaaatcagatcccgtccaaagtgcctcccatgcctaccaccatcatcacatcccccagcaagccagccacctgcccagccgggcctgggatgggccaccacaccactggatattcctgggagtcactgctgacaccatctctcccagcagtcttggggtctgggtgggaaacattggtctctaccaggatccctgccccacctctccccaattaagtgccttcacacagcactggtttaatgtttataaacaaaatagagaaactggtttaatgtttataaacaaaatagagaaactttcgcttataaataaaagtagtttgcacagaaatgaaaaaaaaaaaaaaaaaaaaaaaa

SEQ ID NO: 6 - DNA, 832 bp, Homo sapiens
gggatgctggcgcaggtgggcacaggtggcacaggtgggcgcggggttctaggcgggagatcgctcgggcgaaggccaaggctcgggagctggtgcggggggcgcggccccgagagccgcggaagacgccggtgggggagaagcaggtgccagccaagggcgtcgggccggggctggttccgagcgcgcggcctcgggtcacatgctcgctccgggggccccccgcgcccctcccactggcctctgggccgcagccccacggcgcgccggcagccgggggtcaggccgcggtcagagtgcgggcgcgcgccgtcgccgcagggacgctcccggcccacagtcggcccccgggagcccgggttcgagtcccggccctgccgtgatccgctgcgggacccgggccggcccttccctgtcgggcctcagtcctcccaccgtccctggtcttccctagtcggggctcacgcaagggcgggggaggccaccggtgcccactgcgcgtccgcccctcccccgccgccgggagcgcggacgcgcacgcacctggccgccgctgagagcgagggtagcgggcggagccgggaagccccgcccctctccccgcccccgctcccaccgggccaatcagagggggctacgggacccgtgcggggcgtcgcgctgggggcggggcctgcgggggctgaagagcgagccgggcggacgggccgcaggctgtcgggctggggctgaggctgaggctgaggttgaggcggcggcggcggcggccgggtgcccgggacagcgacgcagcgcgccggcggccgcgacagggccagcgagagccccgcag

SEQ ID NO: 7 - DNA, 538 bp, Homo sapiens
ggagccgccgaggaggtgacggcggcagccctggggcggctcggtgccattcactaccatacctgttttacctgaggcgcggccttccaattgccccggctccgctaggaggcgcgcgctccagccgctccgccgcgcggccccgaccgcacttcagtccggggcgaagaaagaaaaggcggccggctcccgccgctccccgtctacccagccgggcccgcgcaggcgcgcgcagctcccgttcccgcggccgcccctcccccaggccgccgggcgcgccgcgggcggggtcggcgcggggggcggagccggcacgggctcggctggggcccgacccgggattagttggtttcggagcggaggagggagccccgaccgtcacgagcgtcgaagagacaaagccgcgtcagggggcccggccggggcgggggagcccggggcttgttggtgccccagcccgcgcggagggcccttcggacccgcgcgccgccactgccgccgccgccgcctcgcaacaggtccgggcggcctcgctctc